SEQUENCE LISTING (3 sequences)

SEQ ID NO: 1 - DNA, 20 bp, Artificial Sequence
  Synthetic sgNTC
aagcctacttcaccggtcgg

SEQ ID NO: 2 - DNA, 20 bp, Artificial Sequence
  Synthetic sgSox6
ttgacggaatgaactgtacg

SEQ ID NO: 3 - DNA, 20 bp, Artificial Sequence
  Synthetic sgSox6.2
agaacacgctttgagaacct

(19) United States
(12) Patent Application Publication
(10) Pub. No.: US 2024/0167030 A1
(43) Pub. Date: May 23, 2024

(54) METHODS AND COMPOSITIONS FOR ACCELERATING OLIGODENDROCYTE MATURATION

(71) Applicant: Case Western Reserve University, Cleveland, OH (US)

(72) Inventors: Paul J. Tesar, Wickliffe, OH (US); Kevin Allan, Cleveland, OH (US); Tyler Miller, Quincy, MA (US); Matthew Elitt, St. Louis, MO (US)

(73) Assignee: Case Western Reserve University, Cleveland, OH (US)

(21) Appl. No.: 18/283,272

(22) PCT Filed: Apr. 1, 2022
(86) PCT No.: PCT/US2022/023099

§ 371 Date: Sep. 21, 2023

Related U.S. Application Data
(60) Provisional application No. 63/170,152, filed on Apr. 2, 2021.

(51) Int. Cl. C12N 15/113; A61K 35/30; C12N 5/079; C12N 15/86

(52) U.S. Cl. CPC C12N 15/113; A61K 35/30; C12N 5/0622; C12N 15/86; C12N 2310/14; C12N 2310/20; C12N 2710/10041; C12N 2740/15043; C12N 2750/14143

(57) ABSTRACT

A method of accelerating cellular maturation is provided, the method including impairing the activity of developmental transcriptional condensates at an intermediate stage of a cell lineage. Specifically, Sox6 gene expression is inhibited to accelerate maturation of oligodendrocyte precursors, such as oligodrendrocyte progenitor cells (OPCs), into fully mature myelin-producing oligodendrocytes.

### REFERENCE TO RELATED APPLICATION

This application claims the benefit of the filing date of U.S. Provisional Patent Application No. 63/170,152, filed on Apr. 2, 2021, the entire contents of which are incorporated herein by reference.

### GOVERNMENT SUPPORT

This invention was made with Government support under Grant Nos. F30HD096784, T32NS077888, and T32GM007250, awarded by the National Institute of Health (NIH). The Government has certain rights in the invention.

### BACKGROUND OF THE INVENTION

Oligodendrocytes are cells in the central nervous system that are critical for brain health and function by producing myelin, which insulates and protects neuronal axons. Oligodendrocytes arise from a progenitor population in the brain called oligodendrocyte progenitor cells (OPCs), which are present in both the adult and developing brain and are responsible for maintaining oligodendrocyte formation in development and disease.

Oligodendrocyte formation is a multi-step process proceeding from OPC, to intermediate pre-myelinating or newly formed oligodendrocytes, to fully mature myelinating oligodendrocytes. Oligodendrocyte formation has been shown to be stalled at the intermediate premyelinating stage in numerous demyelinating diseases, such as multiple sclerosis.

There is a need for novel therapeutics to accelerate maturation of OPCs to myelin-forming oligodendrocyte to treat numerous demyelinating diseases.

### SUMMARY OF THE INVENTION

Embodiments described herein relate to methods and compositions to accelerate formation of fully mature myelinating oligodendrocytes from precursor stages for the treatment of myelin-related disorders using gene therapy or genome engineering.

Thus, in one aspect, the invention provides a method for accelerating cellular maturation of a cell, the method comprising impairing the activity of developmental transcriptional condensates at an intermediate stage of a cell lineage for the cell.

In some embodiments, the cell is an oligodendrocyte.

In some embodiments, the developmental transcriptional condensates are regulated by Sox6.

In some embodiments, the method comprises delivering to the cell an agent to decrease expression of endogenous Sox6.

In some embodiments, the agent is an antisense oligonucleotide (ASO), an siRNA, a CRISPR interference agent (Cas effector enzyme and guide RNA), TALE/zinc finger protein, NgAO, micro RNA, or a coding sequence therefor.

In some embodiments, the method comprises contacting the cell with a delivery vehicle comprising the agent or the coding sequence therefor.

In some embodiments, the delivery vehicle is an AAV vector, an adenoviral vector, or a lentivirus vector.

In some embodiments, the cell is contacted in vitro, in vivo, or ex vivo.

In some embodiments, the method accelerates oligodendrocyte maturation.

In some embodiments, the method enhances myelination.

In another aspect, the invention provides a modified cell generated by any one of the methods of the invention.

In certain embodiments, the cell is accelerated to maturation by impairing the activity of developmental transcriptional condensates at an intermediate stage.

In some embodiments, the cell is an oligodendrocyte.

In some embodiments, the developmental condensates are regulated by Sox6.

In some embodiments, Sox6 expression is inhibited. In some embodiments, Sox6 expression is inhibited by an antisense oligonucleotide (ASO), siRNA, CRISPR interference, or micro RNA targeting Sox6

In some embodiments, the cell is selected from a neural stem cell (NSC), oligodendrocyte progenitor cell (OPC), or oligodendrocyte cell.

In some embodiments, the modified cell descended from NSC, OPC or oligodendrocyte cell.

In another aspect, the invention provides a composition comprising a modified cell of the invention.

In certain embodiments, the cell is accelerated to maturation by impairing the activity of developmental transcriptional condensates at an intermediate stage.

In some embodiments, the cell is an oligodendrocyte.

In some embodiments, the developmental condensates are regulated by Sox6.

In some embodiments, Sox6 expression is inhibited. In some embodiments, Sox6 expression is inhibited by an antisense oligonucleotide (ASO), siRNA, CRISPR interference, or micro RNA targeting Sox6.

In another aspect, the invention provides a method of treating a myelin related disorder in a subject, the method comprising administering to the subject an agent that impairs the activity of Sox6 to arrest developmental transcriptional condensates at an intermediate stage of the oligodendrocyte precursor—oligodendrocyte lineage, thereby accelerating or promoting maturation of said oligodendrocyte precursor to myelin-producing oligodendrocytes.

In another aspect, the invention provides a method of treating a myelin related disorder in a subject, the method comprising administering to the subject an oligodendrocyte cell accelerated to maturation by impairing the activity of developmental transcriptional condensates at an intermediate stage, thereby introducing mature and myelin-producing oligodendrocyte cell in the subject.

In some embodiments, the myelin-related disorder is selected from multiple sclerosis (MS), neuromyelitis optica (NMO), transverse myelitis, chronic inflammatory demyelinating polyneuropathy, Guillain-Barre Syndrome, progressive multifocal leukoencephalopathy (PML), encephalomyelitis (EPL), central pontine myelolysis (CPM), adrenoleukodystrophy, Alexander's disease, Pelizaeus Merzbacher disease (PMD), Wallerian Degeneration, optic neuritis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute dissmeminated encephalitis, Marie-Charcot-Tooth disease and Bell's palsy.

In some embodiments, the method alleviates at least one symptom(s) of the subject associated with said myelin related disorder.

In some embodiments, the method restores a function of the subject to at least 30%, 40%, 50%, 60%, 70%, 80%, 90%, or about 100% of a control subject without the myelin related disorder, preferably, the function is motor coordination, locomotion, or axon conduction velocity.

In some embodiments, the cell is selected from the group consisting of a modified NSC, OPC, and oligodendrocyte.

In some embodiments, the subject is a mammal, such as a human.

It should be understood that any one embodiment of the invention, including those described only in the examples or claims, can be combined with one or more additional embodiment(s) of the invention, unless explicitly/expressly disclaimed or otherwise improper.

### BRIEF DESCRIPTION OF THE DRAWINGS

FIGS. 1A-1H show that Sox6 is a dominant transcriptional regulator of the OPC state.

FIG. 1A is a schematic detailing the transcriptional and chromatin profiling of in vitro oligodendrocyte progenitor cells (OPCs) and magnetically purified intermediate oligodendrocytes using the surface marker 01.

FIG. 1B shows hockey stick plots of input-normalized, rank-ordered H3K27Ac signal enrichment with super-enhancers highlighted in OPCs (top) and oligodendrocytes (bottom). Example super-enhancer associated genes are listed and their associated regions are indicated as black circles. Data are presented as 2 biological replicates (two independent batches of OPCs from different mouse strains).

FIG. 1C shows heat maps of H3K27Ac signal in OPCs and oligodendrocytes at constituent enhancers of OPC-specific super enhancer loci (two left panels) and at constituent enhancers of oligodendrocyte-specific super enhancer loci (right two panels). Data are presented as 2 biological replicates (two independent batches of OPCs from different mouse strains).

FIG. 1D is a genome browser view of two replicates of H3K27Ac ChIP-seq of OPCs (top two rows) and oligodendrocytes (bottom two rows) at the locus for cell-type-specific super-enhancer-controlled genes Pdgfra and Plp1. Super-enhancer loci are shaded in gray. Scale bars, 5 Kb.

FIG. 1E is a scatter plot of the inward and outward binding for super-enhancer associated transcription factors in OPCs. Each dot represents a single transcription factor and the top 10 connected transcription factors are indicated. Sox6 is highlighted as the most highly connected transcription factor.

FIG. 1F is a bar graph of the top 20 super-enhancer associated transcription factors ranked by clique fraction, or their representation in transcriptional autoregulatory cliques, in OPCs. Clique fraction equals the number of cliques that includes the transcription factor divided by the total number of cliques within the transcriptional regulatory network. Each bar represents a single transcription factor and the top 10 connected transcription factors are highlighted. Sox6 is highlighted as the most highly connected transcription factor.

FIG. 1G shows representative images of intermediate (O1+) and late (MBP+ in green) oligodendrocytes at day 3 of differentiation from OPCs transfected with non-targeting (siNTC) or Sox6 (siSox6) siRNAs. Nuclei are marked by DAPI. Scale bars, 100 μm.

FIG. 1H shows quantification of the percentage of O1 positive and MBP positive oligodendrocytes of total cells (indicated by total DAPI) from OPCs transfected with non-targeting (siNTC as circles) or Sox6 (siSox6 as triangles) siRNAs at day 3 of differentiation. Data are normalized to siNTC presented as mean±SD from 8 technical replicates (individual wells) from a single experiment.

FIGS. 2A-2H show that OPC and intermediate oligodendrocyte super enhancers are enriched for Sox family motifs.

FIG. 2A shows representative images of immunocytochemistry for OPC markers NKX2-2, OLIG2, A2B5, and SOX10 along with the oligodendrocyte marker MBP and astrocyte marker GFAP for both replicate 1 and replicate 2 OPCs. These replicates represent two independent batches of OPCs from different mouse strains. Nuclei are marked by DAPI.

FIG. 2B shows quantification of OPC markers NKX2-2, OLIG2, A2B5, and SOX10, oligodendrocyte marker MBP, and astrocyte marker GFAP as a percent of total cells indicated by DAPI staining for both replicate 1 and replicate 2 OPCs.

FIG. 2C is a heatmap representation of significantly differentially expressed genes between OPCs and oligodendrocytes shown as row Z-score (log 2FC>2, P-adj<0.001). Columns were sorted by unsupervised hierarchical clustering and rows were ranked based on the fold change of gene expression in oligodendrocytes relative to OPCs. Each column represents an individual and independent RNA-seq sample using replicate 1 OPCs and magnetically purified oligodendrocytes.

FIG. 2D shows gene ontology (GO) analysis of genes that are significantly differentially expressed in OPCs and oligodendrocytes (log 2FC>2, P-adj<0.001). Tables show the rank of the GO term along with −log(p-value).

FIG. 2E is a dendrogram of unsupervised hierarchical clustering of gene expression data from our in vitro OPCs and pre-myelinating oligodendrocytes (indicated by “Tesar”) and publicly available gene expression data from in vivo OPCs, intermediate oligodendrocytes, and myelinating oligodendrocytes datasets (Zhang et al, 2014).

FIGS. 2F-2G are box and whisker plots of change in gene expression (TPM) relative to OPCs for OPC-specific (FIG. 2F) and oligodendrocyte-specific (FIG. 2G) super-enhancer associated genes. Gene expression values were taken from our in vitro OPCs and intermediate oligodendrocytes and publicly available datasets for in vivo OPCs, intermediate oligodendrocytes, and myelinating oligodendrocytes (Zhang et al, 2014). The black line represents the median with the box borders representing the upper and lower quartiles with dots representing statistical outliers. p-values were calculated using the Mann-Whitney test for in vitro plots and Kruskal Wallis One-Way ANOVA with Dunn's multiple comparisons test for in vivo plots.

FIG. 2H is a Venn diagram of the transcription factors expressed by the oligodendrocyte lineage whose motifs were significantly enriched (p-value<1×10⁻¹⁰ under super-enhancers in OPCs and oligodendrocytes.

FIGS. 3A-3I show transcriptional regulatory analysis indicating Sox6 as a dominant transcriptional regulator of the OPC state.

FIG. 3A is a schematic representation of the transcriptional regulatory network analysis pipeline. In brief, expressed super-enhancer associated transcription factors are called, the number of transcription factors that bind to open chromatin defined by ATAC-seq transcription factor loci (inward binding) and number of times a transcription factor binds to open chromatin at other transcription factor loci (outward binding) are tabulated, and lastly the prevalence of a transcription factor in autoregulatory transcription factor circuits (or cliques) is calculated.

FIGS. 3B-3C are bar graphs of the top 50 transcription factors ranked by their total binding (sum of their inward and outward binding) in OPCs (FIG. 3B), and intermediate oligodendrocytes (FIG. 3C). Each bar represents a single transcription factor and the top 10 transcription factors in terms of total binding are highlighted in OPCs and in intermediate oligodendrocytes (lighter color bars on the left of each graph). Sox6 is highlighted as the top transcription factor for total binding in both OPCs and intermediate oligodendrocytes.

FIG. 3D is a scatter plot showing the inward and outward binding for super-enhancer associated transcription factors in intermediate oligodendrocytes. Each dot represents a single transcription factor and the top 10 connected transcription factors are indicated in green. Sox6 is highlighted as the most highly connected transcription factor.

FIG. 3E is a bar graph of the top 20 super-enhancer associated transcription factors ranked by clique fraction, or their representation in transcriptional autoregulatory cliques, in intermediate oligodendrocytes. Clique fraction equals the number of cliques that includes the transcription factor divided by the total number of cliques within the transcriptional regulatory network. Each bar represents a single transcription factor and the top 10 connected transcription factors are highlighted. Sox6 is highlighted as the most highly connected transcription factor.

FIG. 3F is a Venn diagram overlapping the top 20 transcription factors in total binding and clique fraction in OPCs (left ovals) and oligodendrocytes (right ovals). The top nodes specifically in OPCs and oligodendrocytes are listed in the left and right text box, respectively, while nodes shared between cell states are highlighted in the middle text box. Genes are listed in order of their total binding.

FIG. 3G is a genome browser view of two replicates of ATAC-seq, and H3K27Ac ChIP-seq in OPCs (top 2 rows of each dataset) and oligodendrocytes (bottom 2 rows of each dataset) at loci for Sox6. Super-enhancer loci are shaded in gray. Scale bar, 50 Kb.

FIG. 3H is Western blot of SOX6 from OPCs transfected with non-targeting (siNTC) or Sox6-targeting (siSox6) siRNAs. Data represent results using replicate 1 and replicate 2 OPCs.

FIG. 3I depicts quantification of the ratio of SOX6 to B-actin of OPCs transfected with siRNA targeting Sox6 or non-targeting siRNA. Data are presented as the mean using replicate 1 and replicate 2 OPCs.

FIGS. 4A-4F show that phenotypic screen, which reveals that miRNAs that drive oligodendrocyte formation, converge on targeting Sox6.

FIG. 4A is a Venn diagram indicating the overlap between super-enhancer associated miRNAs in OPCs (left oval) and oligodendrocytes (right oval). These miRNAs were associated with super-enhancers using both rep1 and rep2 OPCs.

FIG. 4B is a genome browser view of two replicates of H3K27Ac ChIP-seq in OPCs (top two rows) and oligodendrocytes (bottom two rows) at super-enhancer associated miRNA loci including OPC-specific miR-130 and oligodendrocyte-specific miR-219. Scale bars, 2 Kb and 50 Kb respectively.

FIG. 4C shows the top 5 transcription factors in the extended core circuitry governing early oligodendrocyte formation ranked by their score incorporating total binding, clique fraction, and number of super-enhancer associated miRNAs predicted to target the transcription factor. The score calculations are detailed in the methods section.

FIG. 4D is a primary miRNA mimic screen showing the effect of 1309 miRNAs on percentage of oligodendrocytes (MBP+ cells/total DAPI) formed by OPCs relative to T3 treated OPCs. Each dot represents a single miRNA mimic in a single well of a 96 well plate and each miRNA mimic is present twice in biological duplicate with miRNA hits highlighted. Example miRNA hits and positive control miRNAs (mir-138 and miR-219) are labeled with a bar between replicates.

FIG. 4E shows representative immunocytochemistry images of oligodendrocytes from the primary screen of a top miRNA hit (miR-365-3p), T3 positive control, and negative miRNA mimic control. Nuclei are marked by DAPI. Scale bars, 100 μm.

FIG. 4F is a list of miRNA mimic hits that were called from the primary screen as miRNAs that drive oligodendrocyte formation from OPCs. miRNAs highlighted in red are those predicted to target Sox6. Pie charts indicate the number of miRNAs predicted to target Sox6 (in light gray) to miRNAs that do not target Sox6 (in dark gray) within top miRNA hits compared to their prevalence in the whole miRNA screening library (Vlachos et al, 2015). p-value was calculated using hypergeometric analysis.

FIGS. 5A-5E show that exogenous miRNAs that drive oligodendrocyte formation converge on regulating Sox6.

FIG. 5A is a schematic depicting the procedure for the primary miRNA mimic screen to uncover miRNAs that accelerate oligodendrocyte formation from OPCs.

FIG. 5B shows primary screen positive control (T3) and negative control (non-targeting miRNA mimic) percent oligodendrocyte (MBP+/DAPI) metrics on a per plate basis. Data represent mean±SD from 8 technical replicates (individual wells) per plate.

FIG. 5C is a Venn diagram indicating the overlap between top miRNA mimic hits from the primary screen that drive oligodendrocyte formation and miRNAs that significantly increase in oligodendrocytes relative to OPCs.

FIG. 5D shows enrichment network of pathways of genes predicted to be targeted by top miRNA mimic hits. Each node represents a pathway and the size and color of each node is proportional to the number of genes and statistical significance of the pathway respectively.

FIG. 5E shows pie charts depicting the number of miRNAs predicted to target Sox6 (in black) to miRNAs that do not target Sox6 (in gray) within top miRNA hits compared to their prevalence in the whole miRNA screening library. p-values were calculated using hypergeometric analysis.

FIGS. 6A-6G show that Sox6 re-localizes from OPC super enhancers to form aggregates across gene bodies in intermediate oligodendrocytes.

FIG. 6A is a Venn diagram indicating the overlap between SOX6 peaks in OPCs and intermediate oligodendrocytes.

FIG. 6B shows pie charts indicating intersection of SOX6 peaks with all H3K27Ac peaks (FDR<0.001), typical enhancers, or super enhancers in OPCs or oligodendrocytes. p-values were calculated for typical enhancers or super enhancers compared to intersection with all H3K27Ac peaks using hypergeometric analysis.

FIG. 6C shows aggregate binding of OPC-specific SOX6 peaks (n=12,581) and oligodendrocyte-specific SOX6 peaks (n=2,502) within 3 Kb of the center of each peak in OPCs and oligodendrocytes called by MACS2 (narrow peaks, FDR<0.001) normalized to input.

FIG. 6D is a genome browser view of ATAC-seq, H3K27Ac ChIP-seq, and SOX6 ChIP-seq in OPCs (top row of each dataset) and oligodendrocytes (bottom row of each dataset) at SOX6 super-site associated loci including OPC-specific Pdgfra and Oligodendrocyte-specific Bcas1. SOX6 super-sites are shaded in gray. Scale bars, 5 Kb and 10 Kb respectively.

FIG. 6E shows quantification of the normalized number of transcripts (TPM) for both Pdgfra and Bcas1 in OPCs (left bars), and pre-myelinating oligodendrocytes (right bars). Data represent mean±SD from 3 biological replicates (independent samples) from RNA-seq.

FIG. 6F is a hockey stick plot of SOX6 signal across the gene body of all expressed genes in intermediate oligodendrocytes. Genes highlighted are those called as SOX6 aggregate genes (Sox6 signal greater than 2 fold in intermediate oligodendrocytes compared to input and OPC). Example SOX6 aggregate genes are listed.

FIG. 6G shows aggregate plots of ATAC-seq, H3K27-Ac ChIP-seq, and SOX6 ChIP-seq across the gene bodies of SOX6 aggregate genes (top row) or 100 randomly selected expressed genes (bottom row) and 20 Kb upstream from the transcription start site (TSS) and 20 Kb downstream from the transcription end site (TES). Top lines: intermediate oligodrendrocyte; bottom lines: OPC.

FIGS. 7A-7E show that Sox6 re-localizes from OPC super enhancers to form aggregates across gene bodies in intermediate oligodendrocytes.

FIG. 7A depicts quantification of the normalized number of transcripts (TPM) for Sox6 in vivo OPCs, intermediate oligodendrocytes, and myelinating oligodendrocytes. Data represent mean±SD from 2 biological replicates (independent samples) from publicly available RNA-seq.

FIG. 7B shows Western blot of SOX6 from nuclear lysates of OPCs compared to sorted intermediate oligodendrocytes with B-Actin as a loading control using replicate 1 and replicate 2 OPCs.

FIG. 7C shows known motifs significantly enriched under SOX6 peaks in OPCs and intermediate oligodendrocytes. Charts display the transcription factor name, motif, and p-value ranked in order of significance (# indicates rank out of all 1006 motifs in the analysis).

FIG. 7D is a genome browser view of ATAC-seq, H3K27Ac ChIP-seq, and SOX6 ChIP-seq in OPCs (top row in each dataset) and oligodendrocytes (bottom row in each dataset) at Sox6. Super-enhancer loci are shaded in gray. Scale bar, 50 Kb.

FIG. 7E is a zoomed out genome browser view of SOX6 ChIP-seq in OPCs (top row in each dataset) and oligodendrocytes (bottom row in each dataset) at example SOX6 condensate loci including Bcas1 and Nfasc. SOX6 condensates are shaded in gray. Scale bars, 500 Kb.

FIGS. 8A-8G show that Sox6 forms developmental transcriptional condensates at genes transiently expressed during oligodendrocyte differentiation.

FIG. 8A is a genome browser view of ATAC-seq, H3K27Ac ChIP-seq, and SOX6 ChIP-seq in OPCs (top row in each dataset) and intermediate oligodendrocytes (bottom row in each dataset) at an example SOX6 aggregate gene, Nfasc. Scale bar, 20 Kb.

FIG. 8B shows gene ontology (GO) analysis of genes associated with SOX6 aggregates in intermediate oligodendrocytes. The chart includes curated pathways with their rank based on their respective p-values.

FIG. 8C shows gene set enrichment analysis (GSEA) analysis of the SOX6 aggregate genes in in vitro intermediate oligodendrocytes compared to OPCs demonstrates a significant enrichment of SOX6 aggregate genes in intermediate oligodendrocytes.

FIG. 8D is a heatmap representation of row-normalized expression of SOX6 aggregate genes (TPM) in in vivo OPCs, intermediate oligodendrocytes, and myelinating oligodendrocytes.

FIG. 8E is a box and whisker plot of change in gene expression (TPM) relative to OPCs for SOX6 aggregate genes in in vivo OPCs, intermediate oligodendrocytes, and myelinating oligodendrocytes. The black line represents the median with the box borders representing the upper and lower quartiles with dots representing statistical outliers. p-values were calculated using the Kruskal Wallis One-Way ANOVA with Dunn's multiple comparisons test.

FIG. 8F depicts disorder analysis of SOX6 (UniProt: P40645). The algorithms used were: VSL2, IUPred2, and ANCHOR2. An amino acid score above the dotted line indicates a disordered score greater than 0.5 and that the amino acid sequence is disordered. Sequence is written from N-terminus to C-terminus.

FIG. 8G is a schematic of the domains of the SOX6 protein including a coiled-coil domain and HMG box domain. Amino acid positions are indicated from N-terminus to C-terminus.

FIGS. 9A-9G show that Sox6 forms developmental transcriptional condensates at genes transiently expressed during oligodendrocyte differentiation.

FIG. 9A shows Venn diagrams indicating the overlap between SOX6 aggregate genes (left circle in each diagram) and the top 100 genes induced in oligodendrocytes relative to OPCs (top diagram, right circle) and separately with intermediate oligodendrocyte super-enhancer associated genes (bottom diagram, right circle). p-values were calculated by hypergeometric analysis.

FIG. 9B shows Venn diagrams indicating the overlap between SOX6 aggregate genes (left circle in each diagram) and the top 100 signature genes in in vivo OPCs (left diagram, right circle), intermediate oligodendrocytes (middle diagram, right circle), and myelinating oligodendrocytes (right diagram, right circle). p-values were calculated by hypergeometric analysis.

FIG. 9C is a heatmap representation of row normalized expression of SOX6 aggregate genes (log[average UMI]) from single-cell RNA-seq of the oligodendrocyte lineage in the developing mouse brain. COP=committed oligodendrocyte progenitor, NFOL=newly formed oligodendrocyte, MFOL=myelin forming oligodendrocyte, and MOL=mature oligodendrocytes.

FIG. 9D is a bar graph representation of the normalized Z-scores of the SOX6 condensate genes for each cell cluster from the single-cell RNA-seq of the oligodendrocyte lineage in the developing mouse brain. Abbreviations are the same as in FIG. 9C.

FIG. 9E shows aggregate plots of ATAC-seq, H3K27-Ac ChIP-seq, and SOX6 ChIP-seq across the gene bodies of intermediate oligodendrocyte signature genes and 20 Kb upstream from the transcription start site (TSS) and 20 Kb downstream from the transcription end site (TES). Top lines: OPC; bottom lines: oligodendrocyte.

FIG. 9F is a genome browser view of ATAC-seq, H3K27Ac ChIP-seq, and SOX6 ChIP-seq in OPCs (top row in each dataset) and pre-myelinating oligodendrocytes (bottom row in each dataset) at example SOX6 condensate loci including Fyn, and Gpr17. SOX6 aggregates are shaded in gray. Scale bars, 50 Kb and 5 Kb respectively.

FIG. 9G depicts quantification of the normalized number of transcripts (TPM) for Fyn, and Gpr17 from in vivo OPCs (left), intermediate oligodendrocytes (middle), and myelinating oligodendrocytes (right). Data represent mean±SD from 2 biological replicates (independent samples) from publicly available RNA-seq.

FIGS. 10A-10I show that loss of Sox6 accelerates oligodendrocyte maturation.

FIG. 10A shows Western blot of SOX6 from nuclear lysates of control (sgNTC), and Sox6 knockout OPCs using two different guides (sgSox6 g1 and sgSox6 g2) with B-Actin as a loading control.

FIG. 10B is a volcano plot of differentially expressed genes (Log 2FC greater than 0.5 or less than −0.5 and P-adj <0.05) between control (sgNTC) and Sox6 knockout (combined sgSox6 g1 and sgSox6 g2) oligodendrocytes at day 3 of differentiation. Light gray dots near Log 2FC value 0 are genes not significantly different between conditions. Example genes from in vivo signature gene sets are labeled. Data are from 3 biological replicates per condition (independent samples).

FIG. 10C shows gene set enrichment analysis (GSEA) of the expression of SOX6 aggregate genes between SOX6 knockout (sgSox6 g1 and sgSox6 g2) and control (sgNTC) oligodendrocytes at day 3 of differentiation demonstrates a significant depletion of SOX6 condensate genes in SOX6 knockout oligodendrocytes compared to control oligodendrocytes.

FIG. 10D shows gene set enrichment analyses (GSEA) of in vivo OPC (left), intermediate oligodendrocyte (middle), and myelinating oligodendrocyte (right) signature genes between SOX6 knockout (sgSox6 g1 and sgSox6 g2) and control (sgNTC) oligodendrocytes at day 3 of differentiation. This demonstrates a significant depletion of OPC and intermediate oligodendrocyte genes (shaded areas in the left and middle panels) and enrichment of myelinating oligodendrocyte genes (shaded area in the right panel) in SOX6 knockout oligodendrocytes compared to control oligodendrocytes.

FIG. 10E shows representative immunocytochemistry images of oligodendrocytes (MBP+ in green and O1+ in red) from OPCs transfected with non-targeting (siNTC) or Sox6 (siSox6) siRNAs. Nuclei are marked by DAPI. White boxes and zoomed in images to the right are examples of non-matted oligodendrocytes (a), and matted oligodendrocytes (b). Scale bar, 100 μm.

FIGS. 10F-10G show quantification of the percentage of oligodendrocytes of total cells and percentage of matted oligodendrocytes of all oligodendrocytes for markers O1 (FIG. 10F) and MBP (FIG. 10G) from OPCs transfected with non-targeting (siNTC as circles) or Sox6 (siSox6 as triangles) siRNAs. Data are normalized to siNTC and presented as mean±SD from 6 technical replicates (individual wells) from a single experiment.

FIG. 10H show representative immunocytochemistry images of myelinating oligodendrocytes (MBP+, grey) on microfibers with all cells of the oligodendrocyte lineage labeled with OLIG2 (white).

FIG. 10I depicts quantification of the area of MBP+ myelinating oligodendrocytes normalized to the number of OLIG2+ cells per image. Data are normalized to the no transfection condition and presented as mean±SD from 3 separate microfiber well inserts per condition.

FIGS. 11A-11J show loss of Sox6 precociously activates myelination programs.

FIG. 11A is a volcano plot of differentially expressed genes (Log 2FC greater than 0.5 or less than −0.5 and P-adj <0.05) between control (sgNTC) and Sox6 knockout (combined sgSox6 g1 and sgSox6 g2) OPCs. Light gray dots near Log 2FC value 0 are genes not significantly different between conditions. Example genes from in vivo signature gene sets are labeled. Data are from 3 biological replicates per condition (independent samples), except for sgSox6 g1 OPCs, which has 2 biological replicates.

FIG. 11B depicts quantification of the normalized number of transcripts (TPM) for Sox6, Cspg4 and Sox10 in control sgNTC OPCs (left bars), and SOX6 knockout OPCs sgSox6 g1 (middle bars) and g2 (right bars). Data are normalized to sgNTC OPCs and represent mean±SD from 3 biological replicates (independent samples) from RNA-seq, except for sgSox6 g1 OPCs, which has 2 biological replicates.

FIG. 11C depicts quantification of normalized gene expression (TPM) of signature genes from in vivo OPCs, newly formed oligodendrocytes, and myelinating oligodendrocytes in SOX6 knockout OPCs relative to control OPCs (sgNTC). Data are presented as mean±SEM and p-values were calculated using the one-sample Wilcoxon signed rank test.

FIG. 11D shows quantification of the normalized number of transcripts (TPM) of example SOX6 condensate genes Gpr17, Bcas1, and Fyn and myelinating oligodendrocyte genes Gpr37 and Mobp in control sgNTC oligodendrocytes (left bars), and SOX6 knockout (sgSox6 g1 and g2, middle and right bars, respectively) oligodendrocytes. Data are normalized to sgNTC oligodendrocytes and represent mean±SD from 3 biological replicates (independent samples) from RNA-seq, except for sgSox6 g1 OPCs, which has 2 biological replicates.

FIG. 11E shows that SOX6 knockout oligodendrocytes exhibited a significant increase in mature myelinating oligodendrocyte compared to control (sgNTC).

FIG. 11F shows representative images of intermediate (01+) and late (MBP+) oligodendrocytes at days 2 and 3 of differentiation from OPCs transfected with non-targeting or Sox6 siRNAs. Nuclei are marked by DAPI. Scale bars, 100 μm.

FIG. 11G depicts quantification of the percentage of 01 positive and MBP positive oligodendrocytes of total cells (indicated by total DAPI) from OPCs transfected with non-targeting (siNTC as circles) or Sox6 (siSox6 as triangles) siRNAs at days 1, 2, and 3 of differentiation. Data are presented as mean±SD from 4 technical replicates (individual wells) from a single experiment.

FIG. 11H depicts quantification of the percentage of oligodendrocytes displaying matted O1 and matted MBP from OPCs transfected with non-targeting (siNTC as circles) or Sox6 (siSox6 as triangles) siRNAs at day 2 and 3 of differentiation. Data are presented as mean±SD from blinded quantification of images from 4 technical replicates (individual wells) from a single experiment.

FIG. 11I shows representative immunocytochemistry images of myelinating oligodendrocytes (MBP+) on microfibers with all cells labeled with DAPI at days 5 and 10 during differentiation from OPCs.

FIG. 11J depicts quantification of the percentage of oligodendrocytes (MBP+ cells/total cells DAPI) and total MBP+ area of myelinating oligodendrocytes normalized to the total number of MBP+ cells for sgNTC (circles) and sgSox6 g1 (triangles) at days 5 and 10 of differentiation. Data are normalized to sgNTC and presented as mean±SD from 5 separate microfiber wells per condition.

### DETAILED DESCRIPTION OF THE INVENTION

### Overview

Understanding the developmental regulators that ultimately produce functional cell types is a central goal of biology and medicine. While the mechanisms controlling early differentiation have been well-studied for many important cell lineages, the regulation of subsequent maturation timing remains unknown.

The invention described herein is partly based on the discovery that the timing of cellular maturation is governed by a transient form of transcriptional condensates. During oligodendrocyte development, the transcription factor SOX6 dramatically re-localizes from nearly all super enhancers in oligodendrocyte progenitor cells (OPCs) to form developmental condensates across a small set of gene bodies in immature oligodendrocytes. Genes in condensate loci are highly expressed in immature cells but turn off upon maturation.

Consistent with this, CRISPR-, RNAi, or miRNA-mediated suppression of SOX6 reduced developmental condensate gene activation, and accelerated maturation directly to mature myelinating oligodendrocytes.

The data presented herein collectively demonstrates that Sox6-regulated developmental condensates govern processivity along the continuum of oligodendrocyte formation from OPCs. Thus the invention described herein provides, among other things, methods for modulating biomolecular condensates to control maturation rate during development. The methods of the invention can be used, for example, to accelerate the regeneration of mature cell types in numerous diseases.

### Definitions

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as,” “e.g.,” as used herein are non-limiting and are for illustrative purposes only. “Including” and “including but not limited to” are used interchangeably.

The term “or” as used herein should be understood to mean “and/or,” unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

The phrases “parenteral administration” and “administered parenterally” are
art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

The term “pharmaceutical composition” refers to a formulation containing the disclosed compounds in a form suitable for administration to a subject. In a preferred embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, inhalational, and the like. Dosage forms for the topical or transdermal administration of a compound described herein includes powders, sprays, ointments, pastes, creams, lotions, gels, solutions, patches, nebulized compounds, and inhalants. In a preferred embodiment, the active compound is mixed under sterile conditions with a pharmaceutically acceptable carrier, and with any preservatives, buffers, or propellants that are required.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal such as, but not limited to, myelination disturbances, myelin deficiencies, myelin loss and ineffective myelin repair) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent,” “drug,” “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of an agent (e.g., a composition or genetically modified cells described herein) for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The terms “nucleic acid,” “nucleotide,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

A “functional domain” is a domain of a polypeptide comprising a specific activity. Non-limiting examples of activities that a functional domain may possess are nuclease activity, transcriptional regulatory activity, viral capsid recognition activity and the like.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

In general, “CRISPRs” (Clustered Regularly Interspaced Short Palindromic Repeats), also known as SPIDRs (SPacer Interspersed Direct Repeats), refer a family of DNA loci that are usually specific to a particular bacterial species. The CRISPR locus comprises a distinct class of interspersed short sequence repeats (SSRs) that were recognized in E. coli (Ishino et al. (1987) J. Bacteriol., 169:5429-5433; and Nakata et al., J. Bacteriol. (1989) 171:3553-3556), and associated genes. Similar interspersed SSRs have been identified in Haloferax mediterranei, Streptococcus pyogenes, Anabaena, and Mycobacterium tuberculosis (See, Groenen et al. (1993) Mol. Microbiol., 10:1057-1065; Hoe et al. (1999) Emerg. Infect. Dis., 5:254-263; Masepohl et al. (1996) Biochim. Biophys. Acta 1307:26-30; and Mojica et al. (1995) Mol. Microbiol., 17:85-93). The CRISPR loci typically differ from other SSRs by the structure of the repeats, which have been termed short regularly spaced repeats (SRSRs) (Janssen et al. (2002) OMICS J. Integ. Biol., 6:23-33; and Mojica et al. (2000) Mol. Microbiol., 36:244-246). In general, the repeats are short elements that occur in clusters that are regularly spaced by unique intervening sequences with a substantially constant length (Mojica et al. (2000), supra). Although the repeat sequences are highly conserved between strains, the number of interspersed repeats and the sequences of the spacer regions typically differ from strain to strain (van Embden et al., J. Bacteriol. (2002) 182:2393-2401). CRISPR loci have been identified in more than 40 prokaryotes including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Therms, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

“CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a class 1 type I or type III CRISPR system. In some embodiments, one or more elements of a CRISPR system is derived from a class 2 type II, or type V CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. A sequence or template that may be used for recombination into the targeted locus comprising the target sequences is referred to as an “editing template” or “editing polynucleotide” or “editing sequence.” In aspects of the invention, an exogenous template polynucleotide may be referred to as an editing template. In an aspect of the invention the recombination is homologous recombination.

The invention is further illustrated by the following examples, which are not intended to limit the scope of the claims.

### EXAMPLES

### Example 1 Transcriptional Mechanisms Governing the Processivity of Oligodendrocyte Formation from Progenitors

Cells are equipped with transcriptional blueprints that provide unique instructions to establish cellular identity, maintain homeostasis, and shape how cells respond to extracellular cues from the environment (Enver et al., 2009; Mullen et al., 2011). Delineating these transcriptional networks offers insight to the regulation of pathways and processes crucial to cellular function in normal development and how these go awry in disease (Lee and Young, 2013; Ott et al., 2018). Progenitor populations in particular are equipped with transcriptional networks that have the added responsibility to stabilize or direct cell state during differentiation (Adam et al., 2015; Boyer et al., 2005; Tsankov et al., 2015). While differentiation was previously assumed to be a straight line, it is now appreciated that this complex process (Enver et al., 2009; Iacono et al., 2019; MacLean et al., 2018). In particular, interrogating the transcriptional circuitry governing the rate of maturation along a differentiation trajectory provides novel insight to the lineage of interest, and more broadly gives clues to how cellular differentiation is controlled in development and disease.

In the central nervous system (CNS), the oligodendrocyte lineage undergoes a multi-step differentiation process and the transcriptional mechanisms governing the rate of transitioning between states remain poorly understood (Elbaz and Popko, 2019; Emery, 2010; Emery and Lu, 2015; Monje, 2018). Mature myelinating oligodendrocytes are responsible for wrapping neuronal axons in myelin, which is critical for proper action potential propagation and providing neurons with trophic support (Nave, 2010). These mature oligodendrocytes arise from a progenitor population in the brain called oligodendrocyte progenitor cells (OPCs), but first go through an intermediate pre-myelinating oligodendrocyte state (Emery and Lu, 2015; Howng et al., 2010; Zhang et al., 2014). The importance of understanding this differentiation trajectory is highlighted by the numerous diseases of oligodendrocytes, some of which have been shown to impair the lineage at specific states (Allan et al., 2020; Elitt et al., 2018; Gibson et al., 2019; Mathys et al., 2019; Yeung et al., 2019). Knowledge of this differentiation process also provides targets for rational drug design to drive myelin formation in a disease context specific manner (Allan et al., 2020; Elitt et al., 2018; Wang et al., 2020).

Most knowledge of the transcriptional programs regulating oligodendrocyte formation is derived from gene expression data or known disease-causing mutations that lead to hypo-myelinating phenotypes (Emery and Lu, 2015; He et al., 2016; Howng et al., 2010; Sun et al., 2018; Zhou et al., 2001). While effective, an alternative approach to investigate the core circuitry of cell states is through elucidation of super-enhancers, which are chromatin regions enriched for master lineage transcription factors and harbor high levels of transcription of genes critical for cellular identity (Adam et al., 2015; Gryder et al., 2017; Hnisz et al., 2013; Ott et al., 2018; Whyte et al., 2013). In this study, super-enhancer profiling was performed with extended transcriptional network analysis to elucidate the transcriptional mechanisms governing the processivity of oligodendrocyte formation from their progenitors.

### Materials and Methods

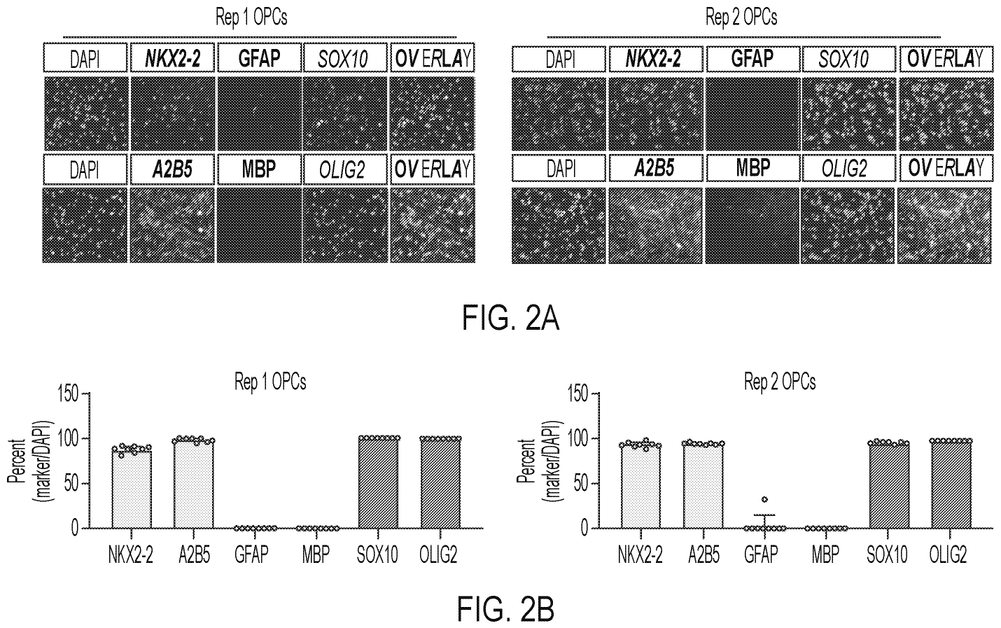

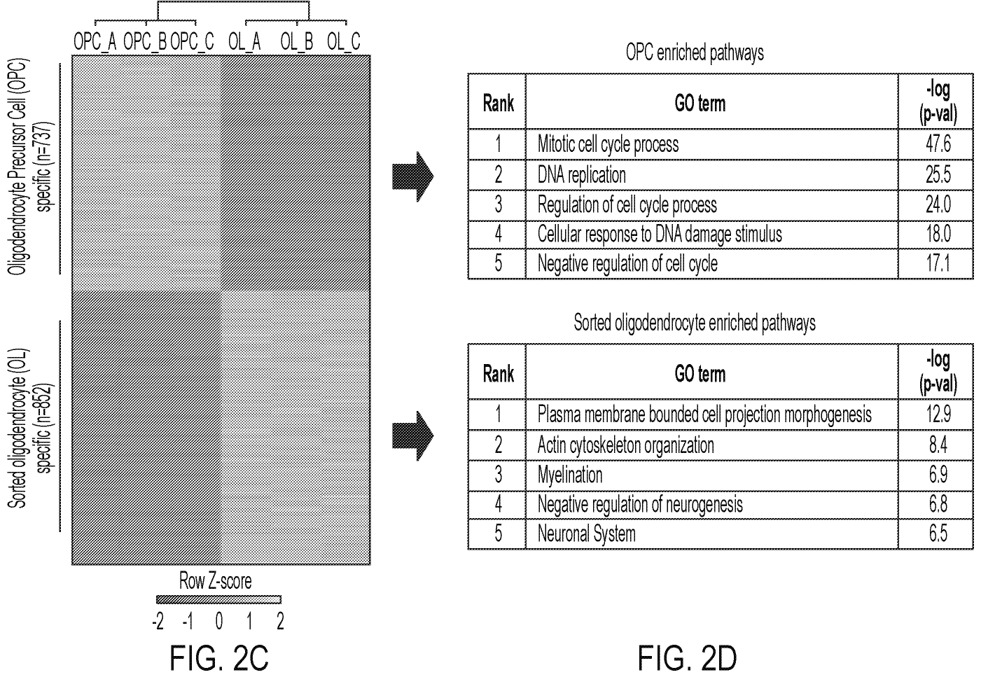

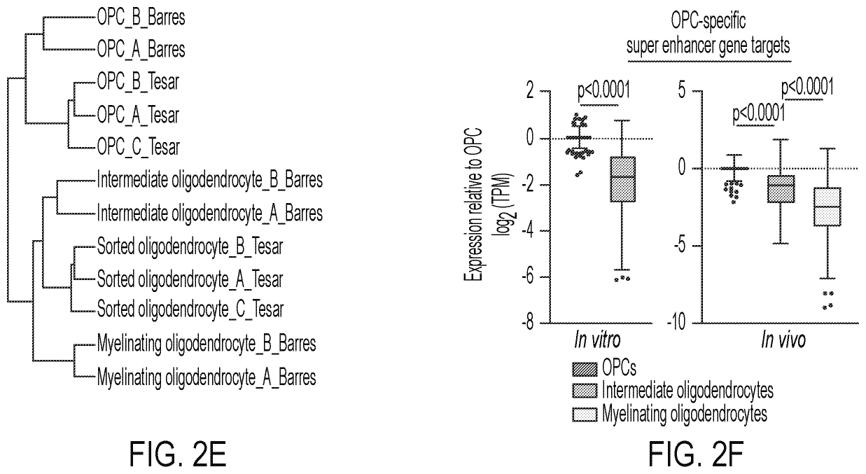

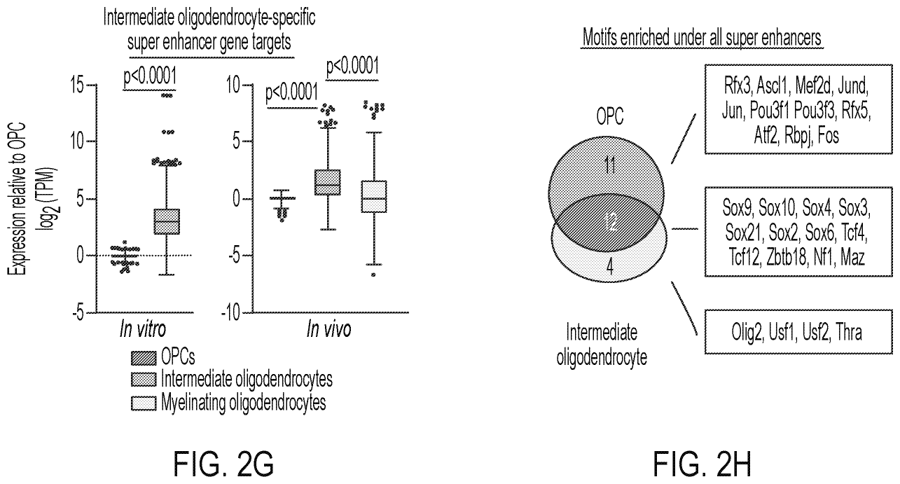

Mouse epiblast derived OPC preparation and culture: Data were generated using OPCs generated from epiblast stem cell (EpiSC) lines EpiSC5 (rep 1 OPCs) and 12901 (rep 2 OPCs), which were derived from mouse strain 129SvEv and 129S1/SvImJ respectively and both have been used and validated in multiple publications (Allan et al., 2020; Hubler et al., 2018; Najm et al., 2015; Najm et al., 2011). These EpiSC derived OPCs were purified by fluorescence activated cell sorting using conjugated CD140a (eBioscience, 17-1401; 1:80) and NG2-AF488 (Millipore, AB5320A4; 1:100) antibodies and purity was verified by staining for markers of OPCs, astrocytes, and oligodendrocytes (FIGS. 2A-2H). Sorted OPCs were then expanded and frozen down in aliquots. All cell and tissue cultures were maintained at 37° C. with 5% CO2 in a humidified chamber. All OPC, oligodendrocyte, and astrocyte cultures were maintained on plates with coated with 100 μg/mL poly(L-ornithine) (P3655, Sigma), followed by 10 μg/ml laminin (L2020, Sigma). Plates were also purchased with a poly(D-lysine) which substituted for poly(L-ornithine). OPC growth media consisted of DMEM/F12 supplemented with N₂ Max (R&D systems), B27 (ThermoFisher), 20 ng/mL bFGF (Fisher), and 20 ng/mL PDGFA (Fisher) and was changed every 48 hours.

Mouse OPC differentiation to oligodendrocytes: OPC differentiation to oligodendrocytes follows the same protocol used previously (Allan et al., 2020; Hubler et al., 2018). In brief, 96 well and 384 well plates were coated with PO (or pre-coated with poly-D lysine) and laminin. OPCs were then seeded at either 40,000 cells per well (96-well plate) or 15,000 cells per well (384-well plate) in oligodendrocyte differentiation media consisting of DMEM/F12 supplemented with N₂ Max (R&D systems), B27 (ThermoFisher), 100 ng/mL noggin, 100 ng/mL IGF-1, 10 uM cyclic AMP, 10 ng/mL NT3 and 40 ng/mL T3. Cultures typically were allowed to proceed for 3 days unless otherwise noted.

Mouse oligodendrocyte myelination assay using microfibers: Parallel-aligned 2-4 μm electrospun fibers composed of poly-L-lactic acid were synthesized and suspended by fitting into 12-well plate inserts by The Electrospinning Company. Inserts were placed in 12 well plates. Prior to use, inserts were incubated in 70% ethanol. Next, fibers were coated with 100 μg/mL poly(L-ornithine) followed by 10 μg/ml laminin. EpiSC5 OPCs seeded at 250,000 OPCs per well in oligodendrocyte differentiation medium. Cells were allowed to attach for 2 hours and then were transfected with 25 nM microRNA mimics, inhibitors, or siRNAs. T3 positive control cells were not transfected. Media was replaced after 16 hours. No other transfection was performed. For T3 positive controls, medium was supplemented with 40 ng/mL thyroid hormone from days 0-3. On day 3 thyroid hormone was removed, and medium was subsequently changed every third day. At day 10 plates were fixed. All plates were immunostained with rat anti-MBP, Olig2 and counterstained with DAPI. Plates were imaged on the Operetta® High Content Imaging and Analysis system. A total of 30 fields were captured at 20× using Acapella® software, and images were analyzed using Harmony® software and Columbus™ software. Using this analysis software we developed an Acapella® script to quantify the total fiber area surrounded by MBP+ oligodendrocytes. All staining, imaging, and analysis steps were performed simultaneously for each microfiber plate using identical procedures to reduce plate-to-plate variability.

Immunohistochemistry: For antigens requiring live staining (01), antibodies were diluted in N2B27 media supplemented with 10% Dokey Serum (017-000-121, Jackson ImmunoResearch) and added to cells in a tissue culture incubator for 18 minutes maintained at 5% CO2 and 37° C. Cells were then fixed by removing either primary live stain solution or cell culture media followed by addition of cold 4% paraformaldehyde (PFA, Electron microscopy sciences). The plates were then incubated at room temperature for 18 minutes, washed with PBS and permeabilized and blocked in blocking solution, which consisted of 0.1% Triton X-100 in PBS supplemented with 10% normal donkey serum (017-000-121, Jackson ImmunoResearch), for at least 30 minutes at room temperature. Primary antibodies (see chart above) were then diluted in blocking solution and incubated on cells overnight at 4° C. The following day, cells were rinsed with PBS and incubated in blocking solution containing appropriate secondary antibodies conjugated to an Alexa-Fluor (ThermoFisher). Secondary antibodies were added at a dilution of 1:500 along with the nuclear stain DAPI (Sigma) at a dilution of 1:10,000.

| | | | | Concentration |
|---|---|---|---|---|
| Antibody Target | Species | Vendor | Cat number | used |
| MBP | Rt | Abcam | ab7349 | 1:100 |
| O1 | Ms | Bob Miller | — | 1:50 |
| GFAP | Rb | Dako | Z0334 | 1:5000 |
| OLIG2 | Rb | Proteintech | 13999-1-AP | 1:500 |
| A2B5 | Ms | Millipore | MAB312 | 1:200 |
| NKX2-2 | Ms | DHSB | 745A5 | 1:200 |
| SOX10 | Gt | R&D | AF2864 | 1:100 |

High content imaging and unbiased quantification using Columbus analysis software: Immunocytochemistry performed on cells in 96-well and 384-well plates were imaged using the Operetta High Content imaging and analysis system (PerkinElmer). Fields were captured at 20× magnification and 8-10 fields (96-well plate) or 5 fields (384-well plate) were captured per well. Images were then uploaded and analyzed with PerkinElmer Harmony and Columbus software as described previously (Allan et al., 2020; Hubler et al., 2018). In brief, total cell number was identified using a threshold for area of DAPI staining of nuclei to exclude pyknotic nuclei and debris. To identify oligodendrocytes, each DAPI nucleus was expanded by 50% and then the intensity of staining of an oligodendrocyte marker (MBP/O1) in a separate channel was calculated. Expanded nuclei that intersected O1/MBP above a threshold were called as oligodendrocytes in that image. The number of oligodendrocytes were then divided by the total number of cells as indicated by DAPI staining to get the percentage of oligodendrocytes per field. All fields were combined to give statistics on a per well basis. Thresholding to call a DAPI positive cell an oligodendrocyte varied on a per-plate basis based on different levels of background, staining intensity, etc. However, the same script is applied to the entire plate to ensure that the same threshold is applied across conditions.

Generation of mouse CRISPR KO OPCs: CRISPR knockout (KO) OPCs were created following a similar protocol used previously (Allan et al., 2020; Tripathi et al., 2019). In brief, guides were selected from the Brie library (Doench et al., 2016), cloned into the linearized CRISPRv2 backbone (Addgene 52961) (Sanjana et al., 2014), and sequence verified by sanger sequencing. HEK293T cells (Clonetech) were than transfected using lenti-X shots (manufacturer's protocol, Clonetech). Transfection media was replaced with N2B27 base media the following day. Lentivirus containing N2B27 was then collected after an additional 2 days, filtered, and supplemented with OPC growth factors PDGFA and FGF2 and added to OPCs at a ratio of 1:2 viral media to OPC growth media. The viral media was switched out for virus-free OPC growth the next day and allowed to recover for an additional 2 days. At this point, infected OPCs were selected for 96 hours in OPC growth media supplemented with a lethal dose of puromcyin (500 ng/mL). OPCs were allowed to recover in selection free media for at least 24 hours prior to being aliquoted and frozen down. For all experiments, infected CRISPR KO and non-targeting control OPCs were derived from the same original batch of EpiSC-derived mouse OPCs. qPCR was performed to validate a reduction of gene targets for each batch of CRISPR KO OPCs generated.

| OPC CRISPR ID | sgRNA sequence (5′ to 3′) |
|---|---|
| sgNTC | AAGCCTACTTCACCGGTCGG |
| sgSox6 | TTGACGGAATGAACTGTACG |
| sgSox6.2 | AGAACACGCTTTGAGAACCT |

Compound screening and validation dose curves: The EpiSC-derived OPC high-throughput screen was performed previously (Factor et al., 2020). In brief, Poly-D lysine coated 384-well CellCarrier ultra plates (PerkinElmer) were coated with laminin diluted in N2B27 base media using a BioTek EL406 Microplate Washer Dispenser (BioTek) equipped with a 5 μl dispense cassette (BioTek). These plates incubated at 37° C. in 5% CO2 for at least an hour. The Selleck bioactives library (X compounds) diluted in dimethylsulfoxide (DMSO) was then dispensed using a 50 nL solid pin tool attached to a Janus automated workstation (Perkin Elmer) at a 1:1000 dilution giving a final concentration of 3 μM per well. OPCs were then plated in OPC differentiation media using the BioTek EL406 Microplate Washer Dispenser. Control columns for this screen included DMSO only as well as a separate column containing FGF2 to impair differentiation and ensure proper response of OPCs to inhibition of differentiation for each plate of the screen. OPCs were then differentiated at 37° C. in 5% CO₂ for 3 days. At the end of the assay, cells were fixed, washed, and stained using the BioTek EL406 Microplate Washer Dispenser. Primary antibody used was MBP (Abcam, ab7349; 1:100) along with Olig2 and DAPI (Sigma; 1 μg/ml) in order to give total cell counts. The plates were imaged using the Operetta High Content Imaging and Analysis system (PerkinElmer). Oligodendrocyte percentage was calculated by taking the number of MBP+ cells and dividing by the number of Olig2+ cells. The oligodendrocyte percentage was normalized between plates. Differentiation inhibitors were called.

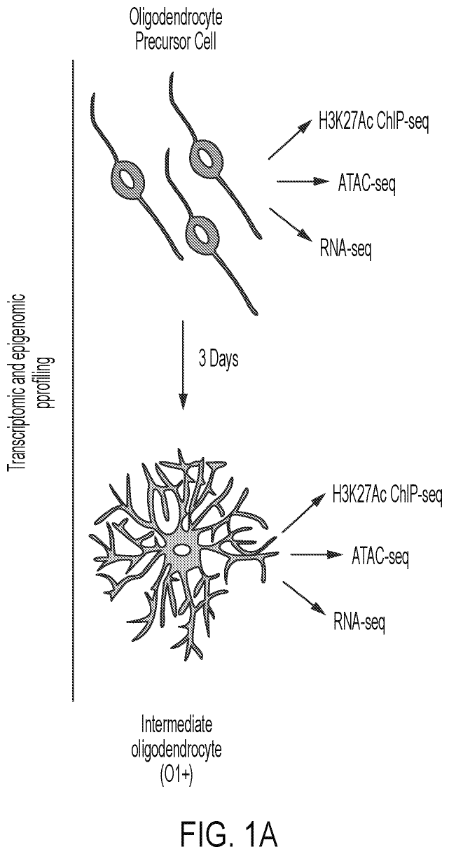

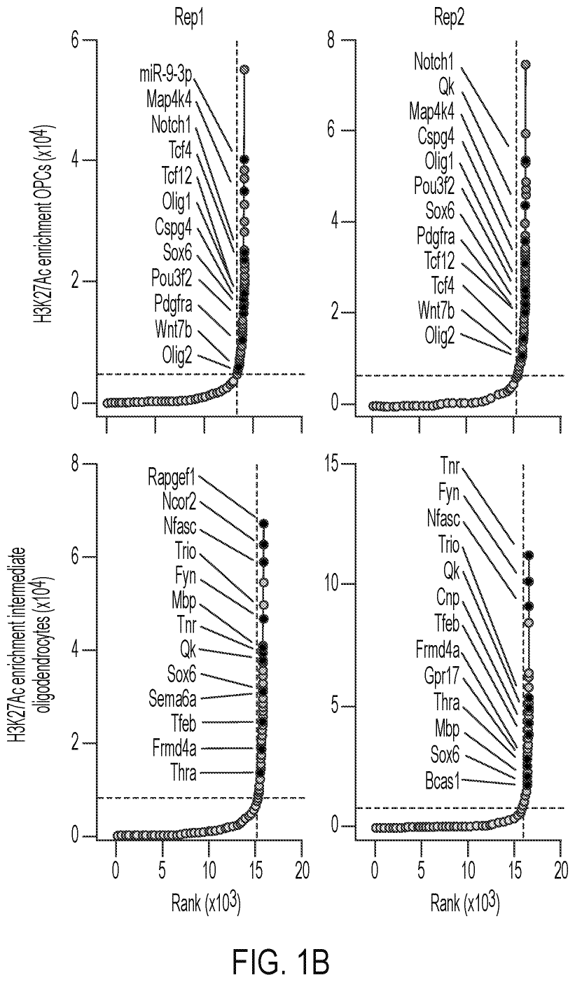

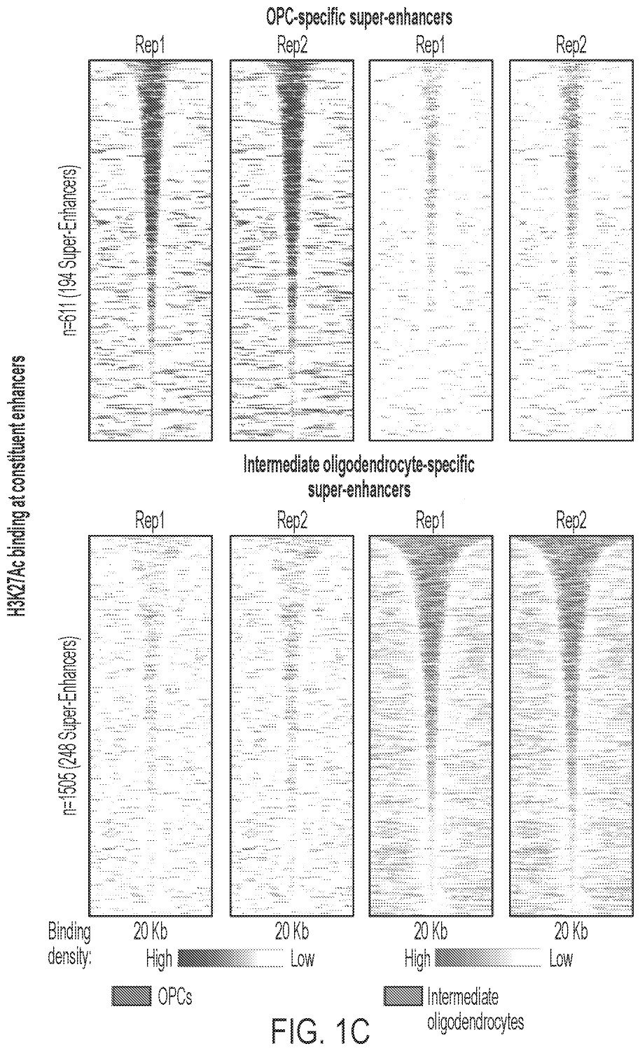

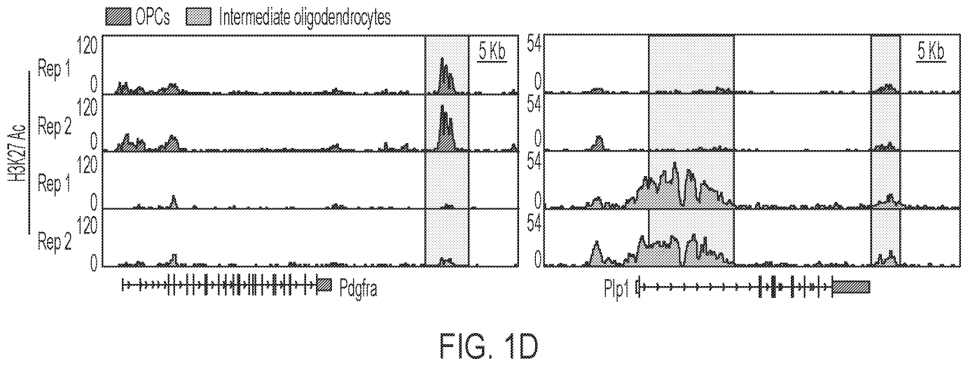

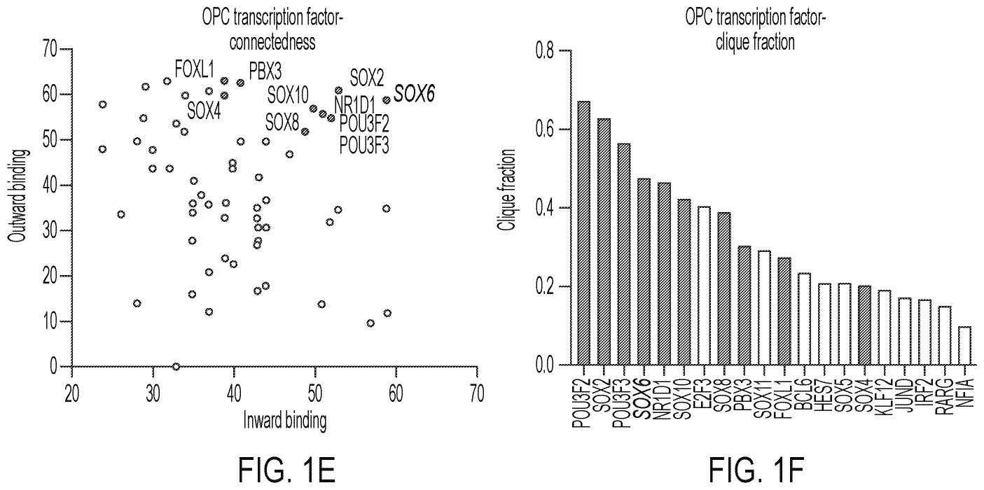

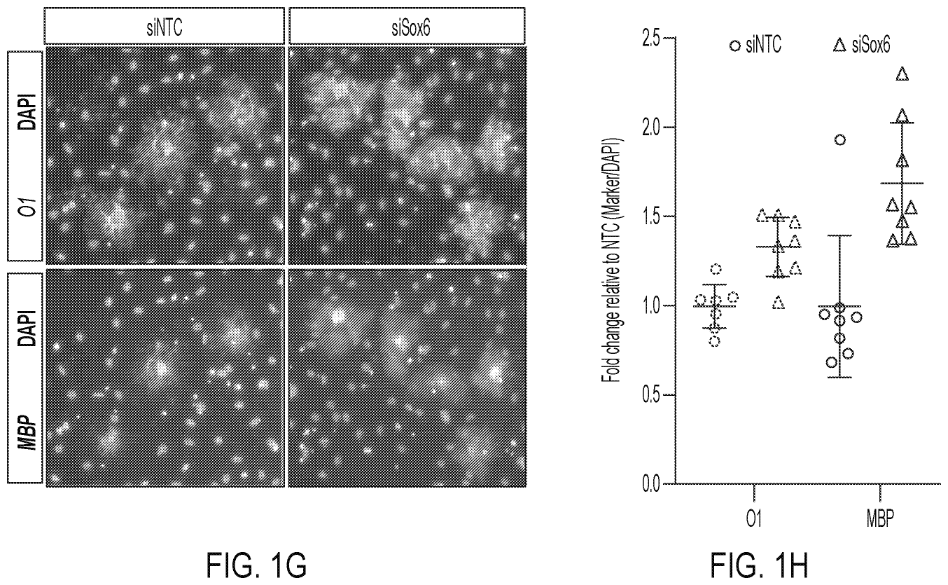

Dose curve plates (FIGS. 1A-1H) were generated by the Small Molecule Drug Discovery core at Case Western Reserve University. In brief, molecules targeting HDACs, CDK7, or BRD proteins were curated from either the bioactives library or a kind gift from Peter Schacheri's lab (THZ1, CT7001). Selected compounds were put into a 10-point dose curve from 10 mM to 3 uM through log-fold dilutions. This was then pinned using the 50 nL solid pin tool of the Janus automated workstation (PerkinElmer) at a 1:1000 dilution into 384 well plates of differentiating OPCs. These cells were allowed to differentiate for 3 days at which point they were fixed and stained for MBP and total cell counts were based on DAPI.

Magnetic sorting to enrich for intermediate oligodendrocytes (O1+): In order to purify pre-myelinating oligodendrocytes for downstream applications such as RNA-seq and ChIP-seq, we performed magnetic sorting using an antibody against O1 (Zhang et al., 2014) (Bob Miller, 1:100) and followed the manufacturer's protocol for the magnetic anti-mouse IgG secondary beads (Miltenyi, 130-048-402). In brief, OPCs were differentiated in Oligodendrocyte differentiation media for 3 days and OPCs were cultured concurrently in growth media. At the end of 3 days, oligodendrocytes and OPCs were harvested using accutase and live stained in suspension with O1 (1:100 with 10 million cells/mL in N2B27 supplemented with 1:15 BSA fraction V and 1:250 EDTA) for 20 minutes on a rocker at 4° C. Cells were then washed with MACs solution (1:20 BSA Miltenyi, 130-091-376 in autoMACs buffer Miltenyi, 130-091-222), pelleted and resuspended in MACs solution (80 ul per 10 million cells) with 20 ul of magnetic anti-mouse IgG beads per 10 million cells. Cells were then rocked at 4° C. for 20 minutes. Cells were then washed, pelleted and resuspended with MACs solution and then strained using 0.22 um flow tube filter caps to remove any clumps. Cells were then processed by the autoMACs cell sorter using the “Posseld2” sorting option to obtain a positive and flow through population. Oligodendrocytes were harvested from the positively selected cells. OPCs grown in growth media were also put through the autoMACs and the negative flow through was used as the OPC population. Enrichment was verified by RNA-seq, demonstrating an enrichment for oligodendrocyte and myelination pathways in the O1 positive population and enrichment for proliferation and stem cell pathways in the OPC population.

ChIP-seq and alignment to genome: OPCs and purified intermediate O1+ oligodendrocytes were harvested using the autoMACs automated cell sorter as described above. Fixation, nuclei isolation and chromatin shearing were performed as previously described using the Covaris TruChIP protocol following the manufacturer's instructions for the “high-cell” format (Allan et al., 2020). In brief, 5 million OPCs and sorted 01+ oligodendrocytes were used for H3K27Ac ChIP-seq whereas 60-90 million OPCs and sorted 01+ oligodendrocytes were used for Sox6 ChIP-seq. Cells were fixed at room temperature for 10 minutes and then immediately proceeded for nuclei extraction. Isolated nuclei were then sonicated using the Covaris S2 with 5% Duty factor, 4 intensity and 4 60-second cycles. Sheared chromatin was then incubated with protein G magnetic DynaBeads (Thermofisher) that were pre-incubated with primary ChIP-grade antibodies overnight at 4° C. Beads were then washed and ChIP DNA was eluted, purified and used to construct illumina sequencing libraries that were submitted to UChicago's genomics core and sequenced on the HiSeq2500 with single-end 50 bp reads with at least 20 million reads per sample. For aligning reads to the genome (mouse, mm10), reads were quality and adapter trimmed using Trim Galore! Version 0.3.1. Trimmed reads were aligned to the mouse genome (mm10) with Bowtie2 version 2.3.2 and duplicate reads were removed using Picard MarkDuplicates. Peaks were called with MACSv2.1.1 with an FDR<0.001 to define broad peaks for histone marks (H3K27Ac) and narrow peaks for transcription factors (Sox6) and normalized to background input genomic DNA. Peaks were visualized using the Interactive Genomics Viewer (IGV, Broad Institute).

| | | | | Amount used |
|---|---|---|---|---|
| Antibody Target | Species | Vendor | Cat number | per ChIP |
| H3K27Ac | Rb | Abcam | ab4729 | 9 ug |
| SOX6 | Rb | Abcam | ab30455 | 15 ug |

Super enhancer analysis: Super enhancers (SE) for OPCs and O1+ sorted oligodendrocytes were called using the ROSE algorithm on H3K27Ac ChIP-Seq data. Super enhancers were called separately for each biological replicate. Gene targets of these SEs were called. miRNA targets of these SEs were called. Super enhancer controlled genes or miRNAs were overlapped between biological replicates 1 and 2 for both OPCs and O1+ sorted oligodendrocytes in order to obtain common SE controlled genes and miRNAs between both replicates.

Omni ATAC-seq: Omni ATAC-Seq was performed on 50,000 OPCs and sorted O1+ oligodendrocytes based on available protocols (Corces et al., 2017). In brief, nuclei were extracted from cells and treated with transposition mixture containing Nextera Tn5 Transposase for (Illumina, FC-121-1030). Transposed fragments were then purified using Qiagen MinElute columns (Qiagen, 28004), PCR amplified, and libraries were purified with Agencourt AMPure XP magnetic beads (Beckman Coulter) with a sample to bead ratio of 1:1.2. Samples were submitted to UChicago's genomics core (https://fgf.uchicago.edu/) and sequenced on the HiSeq2500 with single-end 50 bp reads with nearly 100 million reads per sample. Reads were aligned to the mm10 mouse genome following the same pipeline that was used for aligning ChIP-seq data and peaks were called using the same pipeline for H3K27Ac ChIP-seq.

Motif enrichment analysis: Motifs were called under significant Sox6 peaks (FDR<0.001) or ATAC-Seq peaks (FDR<0.001) within super enhancers using HOMERv4.11.1 (Adam et al., 2015; Heinz et al., 2010). The FindMotifsGenome.pl tool was used with 200 bp windows for ATAC-Seq regions and Sox6 peaks using mm10 as the reference genome.

Western blot: For cell culture derived protein samples, at least 1 million OPCs were collected and lysed in RIPA buffer (Sigma) supplemented with protease and phosphatase inhibitor (78441, Thermo Fisher) for at least 15 minutes and cleared by centrifugation at 13,000 g at 4° C. for 15 minutes. Protein concentrations were determined using the Bradford assay (Bio-Rad Laboratories). Protein was diluted, boiled at 95° C. for 5 minutes, run using NuPAGE Bis-Tris gels (NP0335BOX, Thermo Fisher), and then transferred to PVDF membranes (LC2002, Thermo Fisher). Membranes incubated with primary antibodies at 4° C. overnight in blocking solution consisting of 5% nonfat drug milk (Nestle Carnation) in TBS plus 0.1% Tween 20 (TBST). Primary antibodies used included Sox6 (1 μg/mL, Abcam, ab30455) and B-Actin peroxidase (1:50,000, Sigma, A3854). Membranes were then imaged and analyzed using Image Studio™ software. Westerns were normalized to the Beta-Actin loading control unless otherwise noted.

qPCR: OPCs were lysed in TRIzol (Ambion) followed by purification and elution of RNA using phenol-chloroform extraction and the RNeasy Mini Kit (74104, Qiagen). RNA quality and quantity were determined using a NanoDrop spectrophotometer. cDNA was generated using the iSCRIPT kit following the manufacturer's instructions (1708891, Biorad). qRT-PCR was performed using pre-designed TaqMan gene expression assays (Thermo Fisher) as detailed in the chart below. qPCR was performed using the Applied Biosystems 7300 real-time PCR system and probes were normalized to Rp113a endogenous control.

| Gene ID | Taqman cat. number |
|---|---|
| Sox6 | Mm00488393_m1 |
| Rpl13a | Mm05910660_g1 |

Bulk RNA-seq sample preparation and alignment: OPCs were lysed in TRIzol and RNA was isolated as described for qPCR. Purified RNA was then submitted to Novogene for library preparation and sequencing on the Illumina NovaSeq (150 bp paired end). For gene expression analysis, reads were aligned to the mm10 genome and quantified in transcripts per million (TPM) values using salmon 0.14.1 (github dot com slash COMBINE-lab slash salmon). Transcripts were summarized as gene-level TPM abundances with tximport. A gene with TPM>1 was considered expressed. Differential expression analysis was performed using DESEQ2 (bioconductor.org slash packages slash release slash bioc slash html slash DESeq2 dot html). Significant genes were called based on p-adj and fold change values as described in the results section.

Gene ontology analysis: Metascape (Zhou et al., 2019) was used to identify significant pathways from gene lists called using fold change and significance (p-adj) cut-offs described in the results section.

Statistics and replicates: GraphPad Prism was used to perform statistical analyses unless otherwise noted. Statistical tests and replicate descriptions are detailed in each figure legend. Black filled-in circles for bar graphs indicate biological replicates (independent experiments) whereas open circles represent technical replicates. Statistics were only performed on samples with biological replicates. Data is typically graphed as mean±standard deviation (SD) or ±standard error of the mean (SEM) as detailed in the figure legend. A p-value less than 0.05 was considered significant unless otherwise noted.

### Example 2 Super-Enhancers in OPCs and Intermediate Oligodendrocytes are Enriched for SOX Family Motifs

The core transcriptional networks regulating cell state can be defined by super-enhancers, which are large regions of active chromatin densely packed with transcriptional machinery to ultimately regulate genes and pathways critical for cellular identity and function (Hnisz et al., 2013; Whyte et al., 2013). Utilizing scalable two independent batches of scalable and pure populations of mouse pluripotent stem cell-derived OPCs, RNA sequencing (RNA-seq), H3K27Ac chromatin immunoprecipitation coupled with sequencing (ChIP-seq), and Assay for Transposase-Accessible Chromatin using sequencing (ATAC-seq) were performed on OPCs and magnetically sorted O1 positive oligodendrocytes (Najm et al., 2011; Corces et al., 2017; Creyghton et al., 2010) (FIGS. 1A, 2A, and 2B). Gene ontology (GO) analysis of differentially expressed genes between OPCs and sorted oligodendrocytes (log 2FC>2, P-adj <0.001) confirmed enrichment for processes indicative of cell state such as proliferation pathways in OPCs and actin cytoskeleton organization in oligodendrocytes (FIGS. 2C and 2D).

Comparing the transcriptional states of in vitro OPCs and oligodendrocytes with in vivo isolated OPCs, intermediate oligodendrocytes, and fully mature myelinating oligodendrocytes revealed that in vitro sorted oligodendrocytes most closely align with intermediate in vivo oligodendrocytes (Zhang et al., 2014) (FIG. 2E). This highlights that this culture system provides unique access to pure and scalable quantities of both OPCs and this intermediate step of mature oligodendrocyte formation.

The Ranking of Super Enhancer (ROSE) algorithm was then performed using H3K27Ac ChIP-seq to call super enhancers in both OPCs and intermediate oligodendrocytes (Adam et al., 2015; Hnisz et al., 2013; Loven et al., 2013) (FIG. 1B). Calling OPC and oligodendrocyte-specific super-enhancers and their associated genes demonstrated that these regions were strongly enriched for H3K27Ac and gene expression in a cell-state-specific manner (FIGS. 1C, 1D, 2F, and 2G).

Performing motif analysis under open chromatin regions defined by ATAC-seq within all super-enhancers of both states revealed an enrichment for OPC-enriched, shared, and oligodendrocyte-enriched transcription factor motifs (FIG. 1H). Transcription factors known to promote cellular proliferation, such as JunD and Fos, were enriched specifically in OPCs, whereas the thyroid hormone receptor, Thra, was specifically enriched in oligodendrocytes, which agrees with the culture system using thyroid hormone (T3) to drive oligodendrocyte formation (Gao et al., 1998; Shaulian and Karin, 2001) (FIG. 2H). Of the motifs shared between both states, more than half were Sox family motifs, suggesting that this transcription factor family plays a crucial role regulating this early differentiation process (FIG. 1H).

### Example 3 Transcriptional Regulatory Network Analysis Elucidates Sox6 as a Dominant Transcriptional Regulator of the OPC State

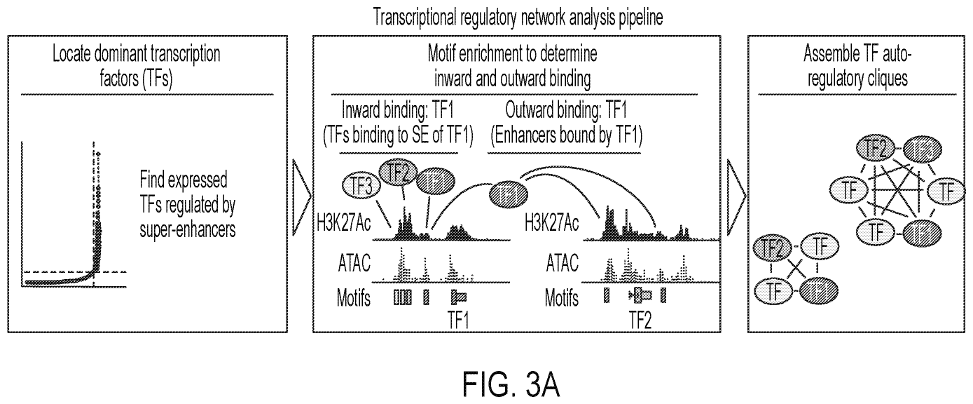

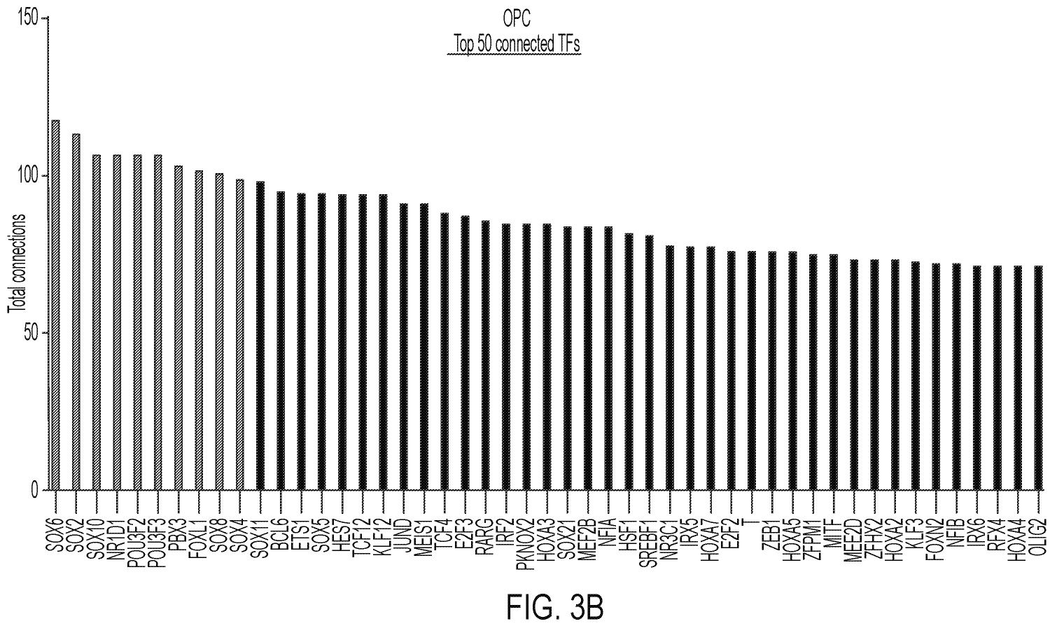

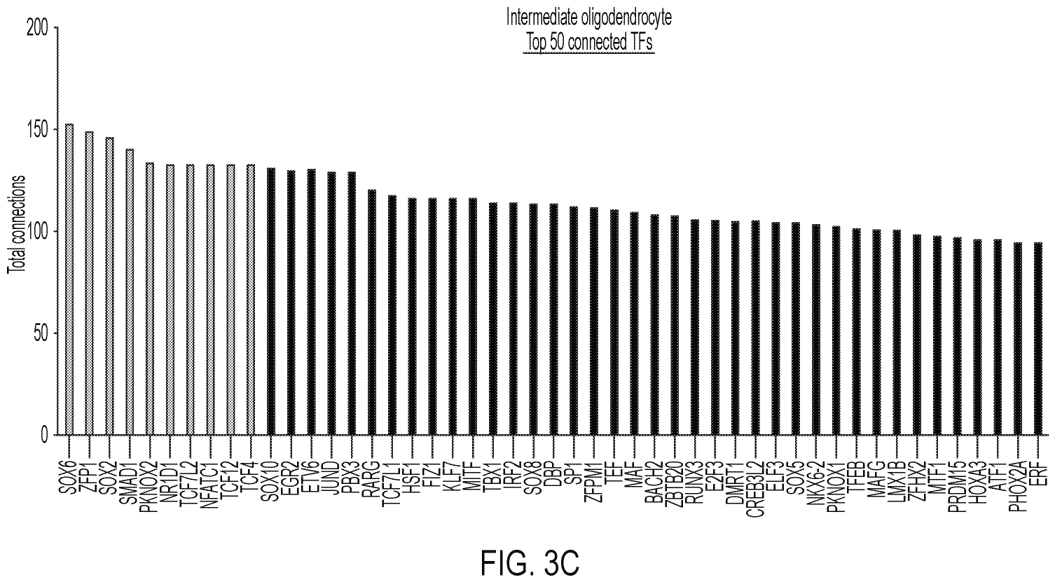

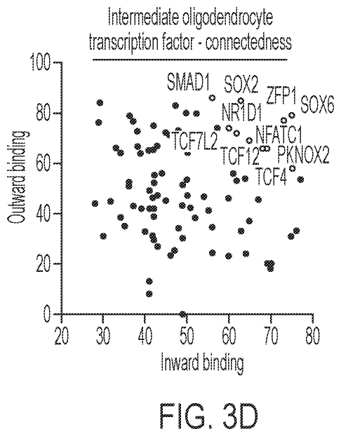

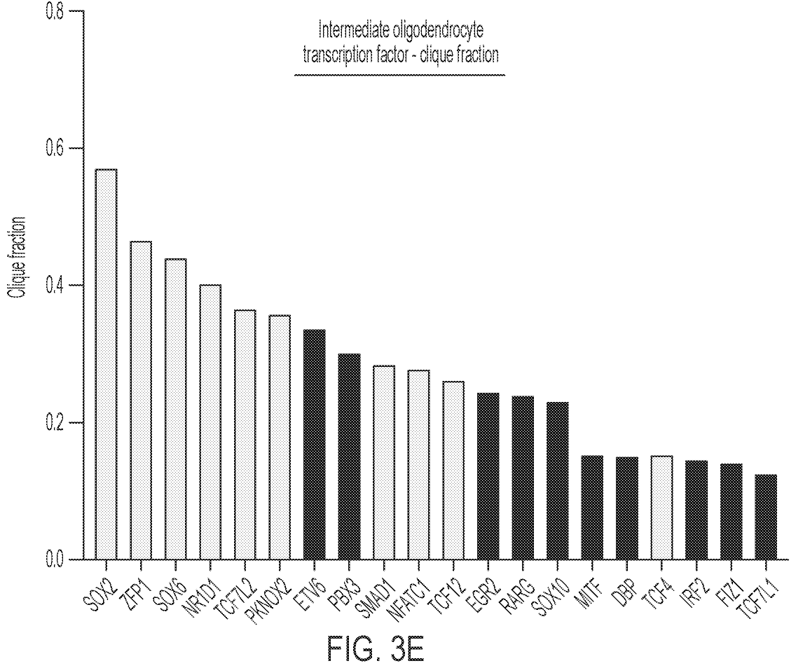

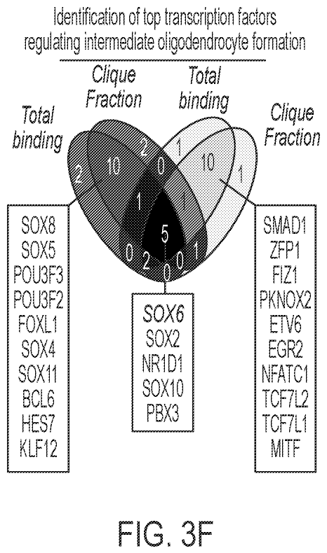

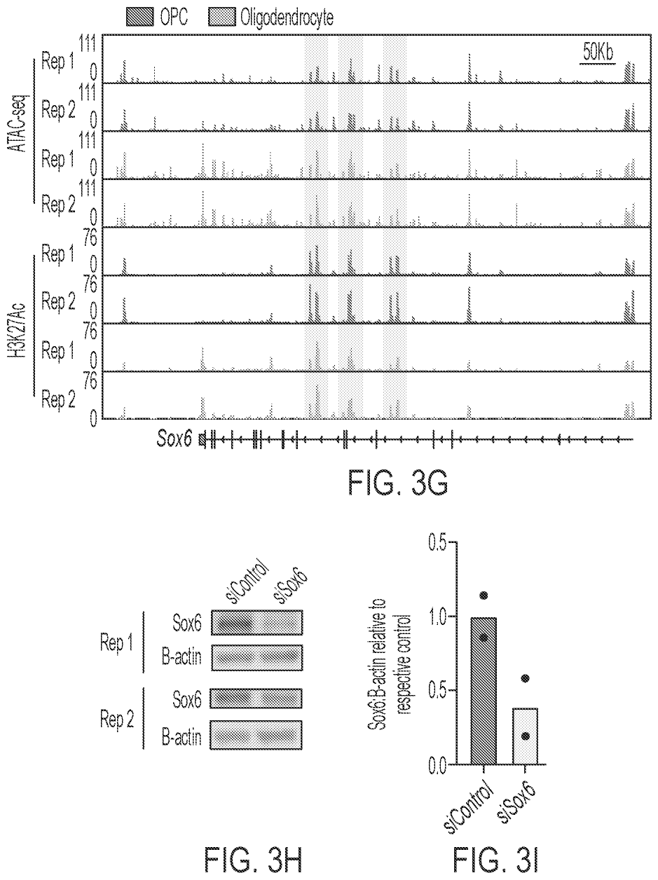

Super-enhancer associated transcription factors play crucial roles regulating the transcriptional network governing cell state (Adam et al., 2015; Mercado et al., 2019). To further parse dominant transcriptional regulators of processivity through the progenitor state, a transcriptional regulatory network analysis approach were utilized to interrogate master regulators of OPC state (Bleu et al., 2019; Federation, 2018; Mercado et al., 2019; Ott et al., 2018) (FIG. 3A). This pipeline tabulated the connectedness of an expressed super-enhancer associated transcription factor within the regulatory network by determining the number of transcription factors predicted to bind at its locus (inward binding) and the number of times it is predicted to bind at other transcription factor loci (outward binding), and lastly the prevalence of the transcription factor motif in self-reinforcing networks (auto-regulatory cliques), a defining aspect of master transcription factors (Boyer et al., 2005; Ott et al., 2018) (FIG. 3A).

Together, these analyses demonstrated that Sox6 is the most connected transcription factor and highly revalent within auto-regulatory cliques in both states (FIGS. 1E, 1F, 3B-3F). This is reflected by the impressive levels of open and active chromatin at the Sox6 locus in both states (FIG. 3G). To assess functionality of this dominant node, Sox6 in OPCs was knocked down, which led to an increase in formation of O1+ and MBP+ oligodendrocytes (FIGS. 1G, 1H, 3H, and 3I). This suggested that the main role of Sox6 is to stabilize the OPC state, which has been suggested by others (Baroti et al., 2016; Emery and Lu, 2015; Stolt et al., 2006).

### Example 4 Extended Transcriptional Network Analysis Identifies Sox6 as the Top Node During Pre-Myelinating Oligodendrocyte Formation

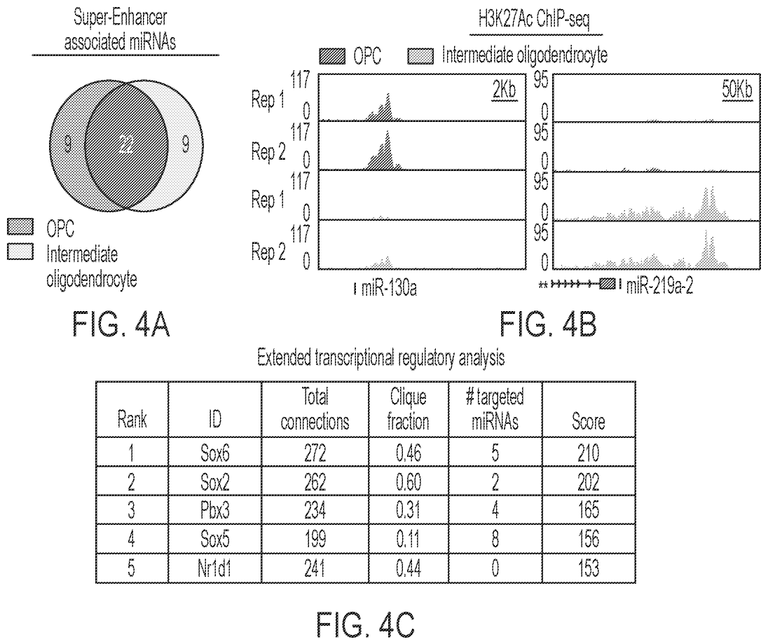

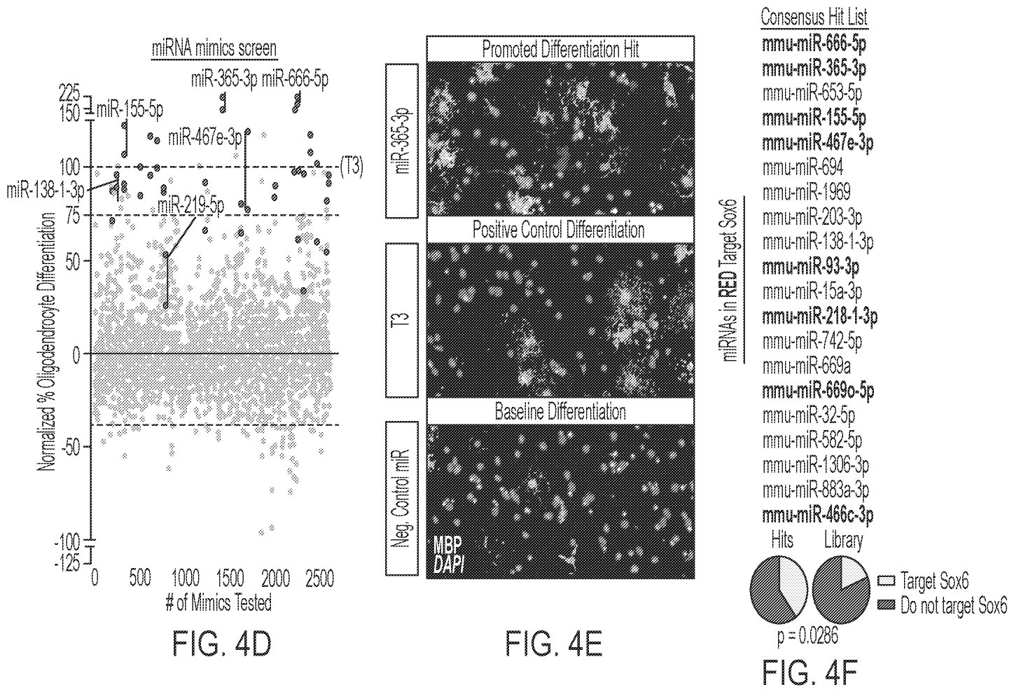

Alongside transcription factors, super-enhancer controlled micro RNAs (miRNAs) provide an additional layer of feedback to regulate the core circuitry governing cell state and critical cellular processes (Marson et al., 2008; Suzuki et al., 2017; Tiscornia and Izpisua Belmonte, 2010). This is underscored by the fact that miRNA processing machinery and multiple miRNAs have been shown to be critical for oligodendrocyte formation from OPCs (Dugas et al., 2010; Emery and Lu, 2015; Zhao et al., 2010). Performing small RNA sequencing coupled with H3K27Ac ChIP-seq, expressed super-enhancer associated miRNAs in both OPCs and oligodendrocytes were called (FIGS. 4A and 4B).

Using an established miRNA target prediction algorithm, how many of these miRNAs predicted to regulate transcription factors in the core transcriptional network (Vlachos et al., 2015) were determined. Combining the connectedness, prevalence in auto-regulatory cliques, and number of predicted targeting super-enhancer associated miRNAs for each transcription factor further highlighted Sox6 as a master regulator of this early state change (FIG. 4C).

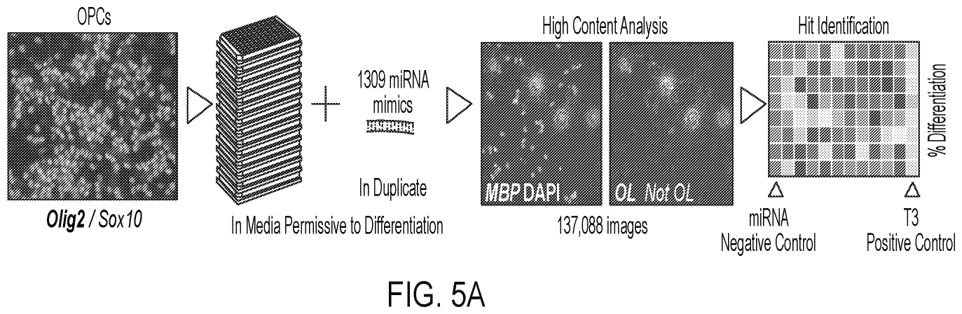

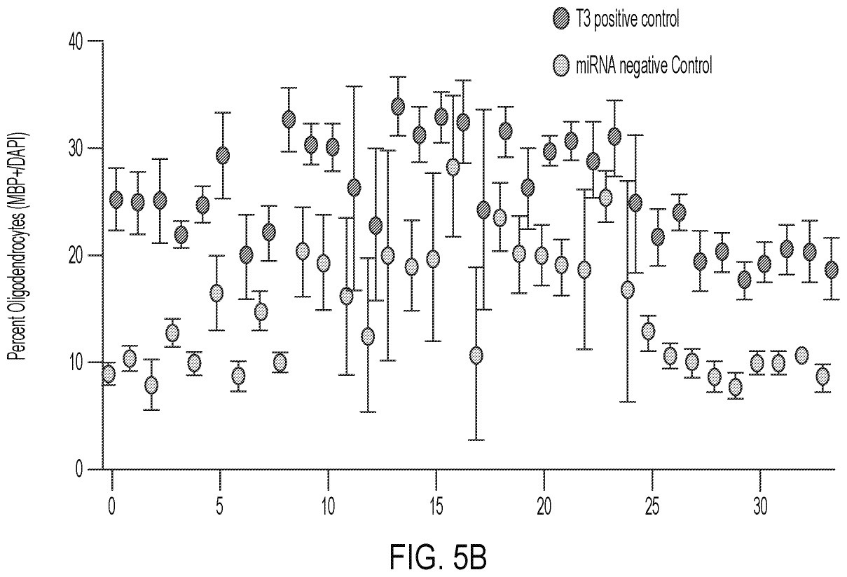

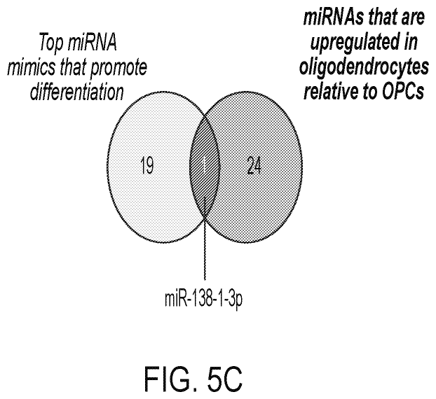

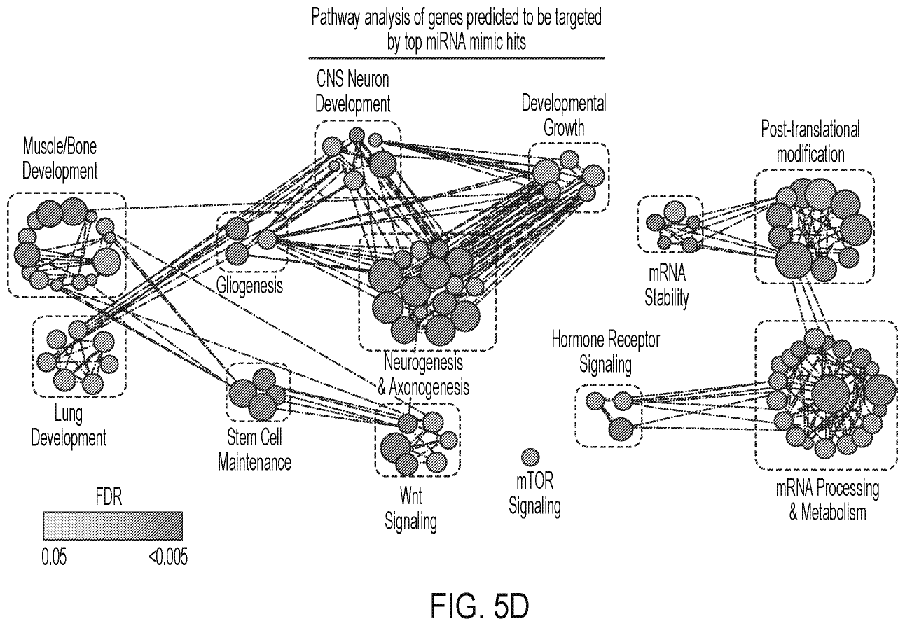

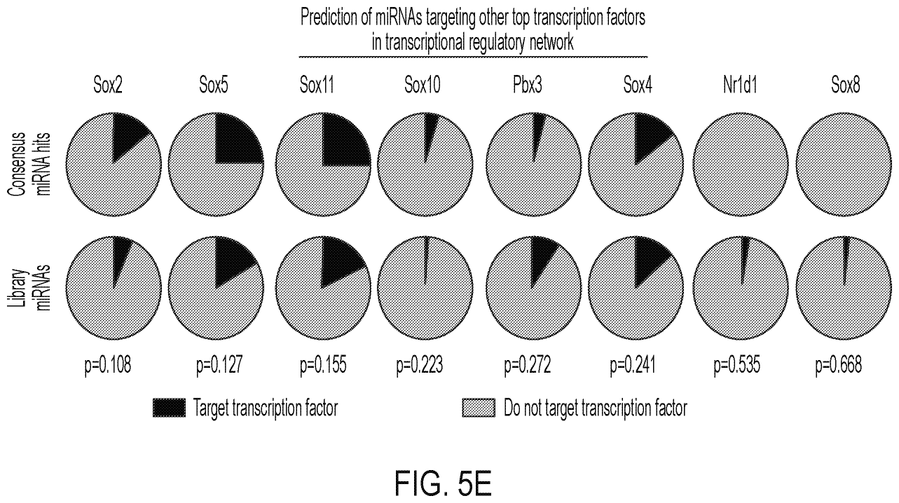

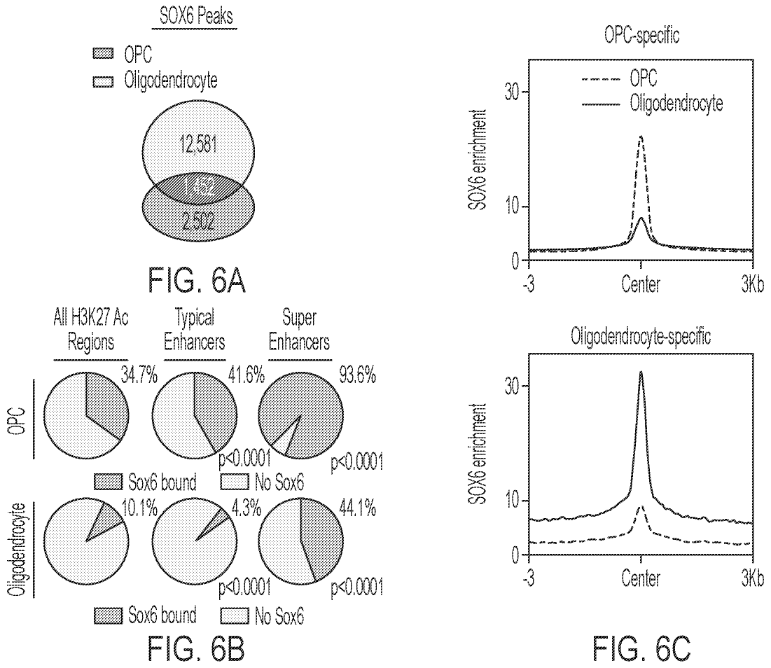

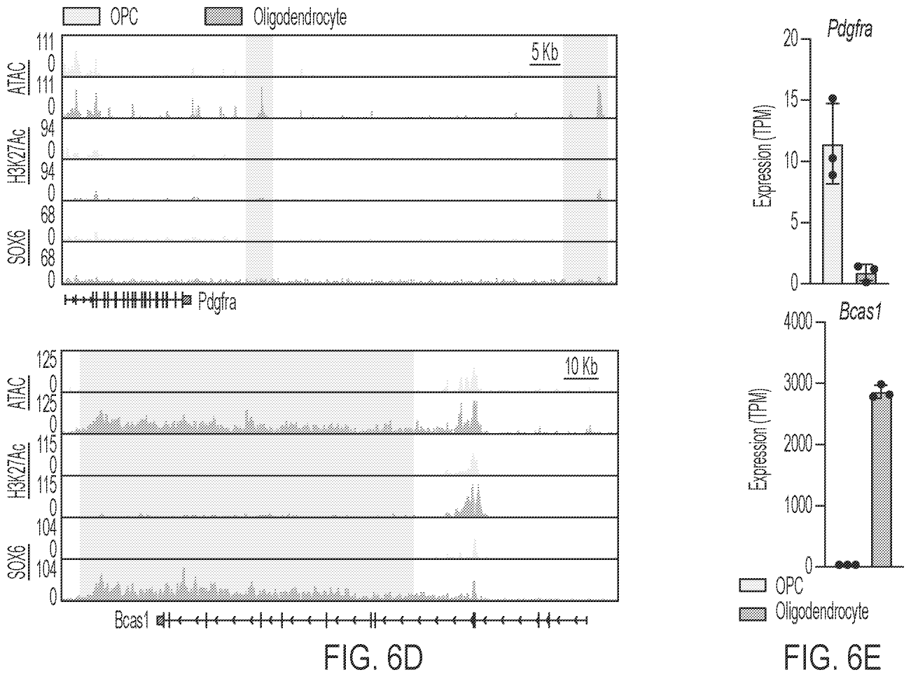

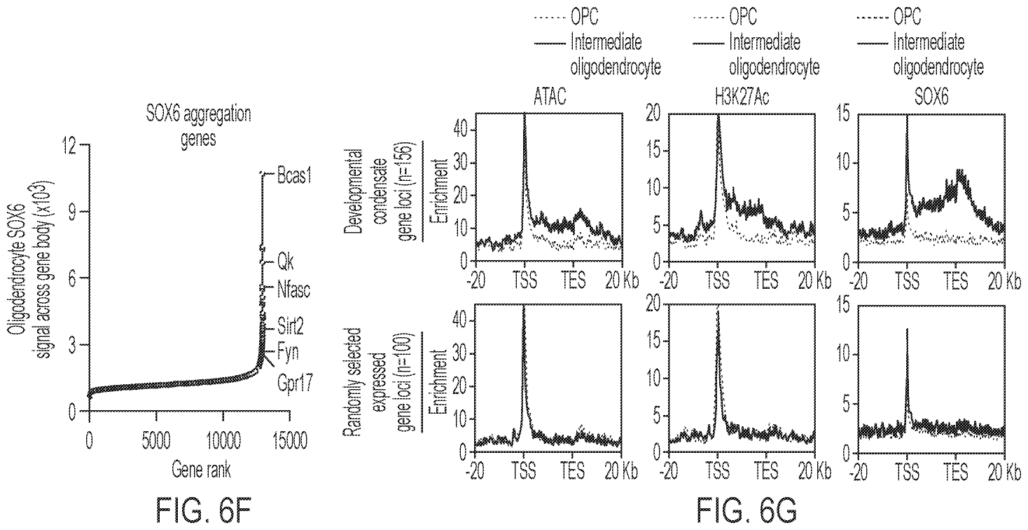

To further unbiasedly uncover miRNAs that interact with the core transcriptional network, miRNAs that are capable of driving the formation of oligodendrocytes from OPCs were screened. Specifically, OPCs were transfected with 1,309 individual miRNA mimics in duplicate followed by high throughput imaging and quantification (FIGS. 4D, 4E, 5A, 5B). miR-219 and miR-138 are known drivers of oligodendrocyte formation in vivo and both led to an increase in oligodendrocyte formation in our screen highlighting the physiological relevance of the primary screen (Dugas et al., 2010; Elbaz and Popko, 2019; Zhao et al., 2010) (FIG. 6A). Interestingly, the majority of the miRNAs with the strongest phenotypic effects on oligodendrocyte formation were not significantly increased in expression in intermediate oligodendrocytes compared to OPCs, and therefore would have been missed if relying solely on expression data (FIG. 5C). Target prediction analysis of the top miRNA mimic hits revealed an enrichment for developmental and neuronal pathways and Sox6 as the only transcription factor from the transcriptional regulatory network that was significantly enriched as a target of miRNA hits compared to all miRNAs in the library (FIGS. 4F, 5D, and 5E).

Taken together, Sox6 is identified as a highly connected transcription factor node in the transcriptional and post-transcriptional networks governing the progenitor state such that loss of Sox6 increases oligodendrocyte formation.

### Example 5 SOX6 is Enriched at Super-Enhancers in OPCs and Re-Localizes to Form Aggregates Across Gene Bodies in Intermediate Oligodendrocytes

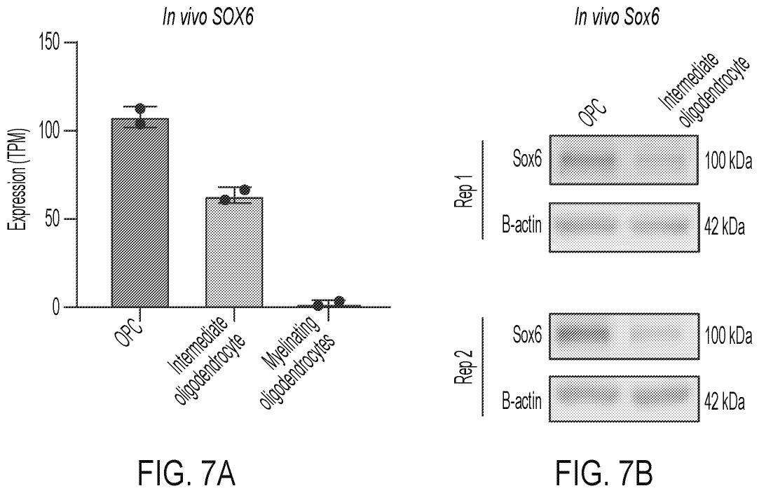

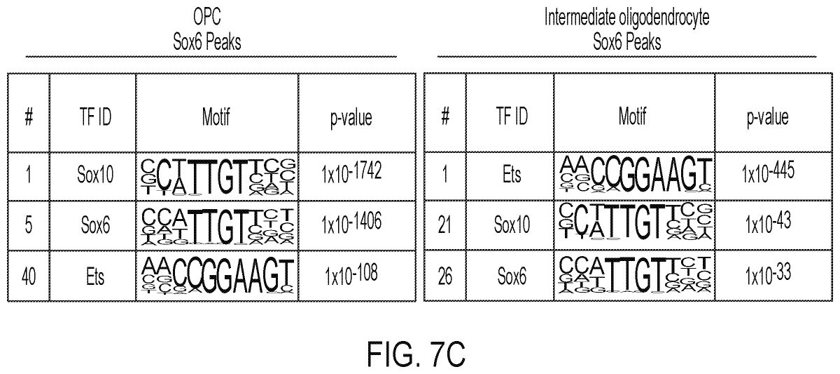

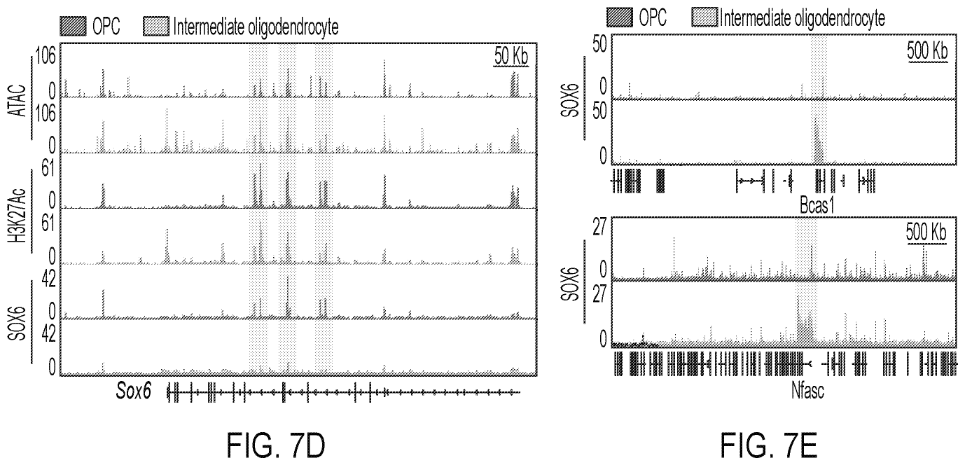

Sox6 expression decreases as OPCs differentiate and is thought to primarily maintain OPCs in the progenitor state by both inhibiting oligodendrocyte genes and driving the expression of the OPC signature gene Pdgfra (Baroti et al., 2016; Emery and Lu, 2015; Stolt et al., 2006). However, Sox6 expression does not completely disappear in intermediate pre-myelinating oligodendrocytes and Sox6 was called as a dominant transcription factor in intermediate oligodendrocytes (FIGS. 2B, 2C, 2E, 7A and 7B). Interrogating the whole genome binding profile of Sox6 in OPCs and intermediate oligodendrocytes could shine light on this discrepancy (FIGS. 4A-4F). Given what is currently known about Sox6, one would hypothesize that Sox6 binds near OPC and oligodendrocyte genes in OPCs and that these peaks disappear in intermediate oligodendrocytes as Sox6 expression declines.

Performing ChIP-seq for SOX6 in OPCs and intermediate oligodendrocytes demonstrated a greater number of peaks in OPCs, which agrees with SOX6 expression levels, and an enrichment for SOX6 at super-enhancers in both states (FIGS. 6A-6B, and 7C). However, intermediate oligodendrocytes harbored a clear gain in state-specific SOX6 peaks suggesting SOX6 does not simply disappear, but instead changes locations during the process of differentiation (FIGS. 6C-6E). SOX6 was also strongly bound to its own locus in OPCs, which agrees with the self-regulating nature of master lineage transcription factors (Boyer et al., 2005) (FIG. 7D). A portion of intermediate oligodendrocyte specific SOX6 sites displayed a particularly interesting binding behavior in which Sox6 signal spread across gene bodies such as Bcas1, which dramatically increases in expression in intermediate oligodendrocytes (FIGS. 6D and 6E). Tabulating the intensity of SOX6 signal across expressed genes in oligodendrocytes revealed 156 associated “Sox6 aggregate genes,” which were similarly enriched for active and open chromatin signatures (FIGS. 6F, 6G, and 7E). Collectively, this suggests that SOX6 re-localizes from super-enhancers in OPCs to gene bodies boasting robust active chromatin signatures.

### Example 6 SOX6 Forms Biomolecular Condensates in Intermediate Oligodendrocytes at Genes Transiently Expressed During Differentiation

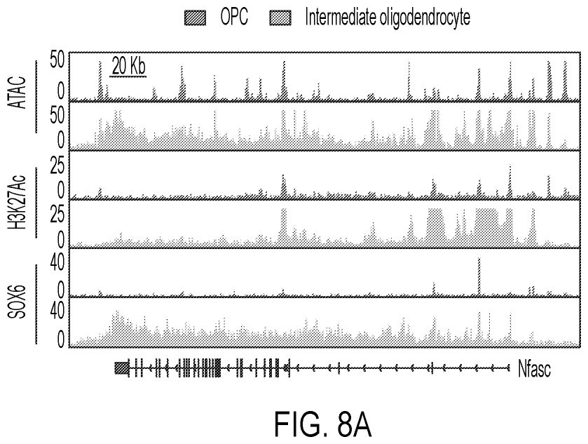

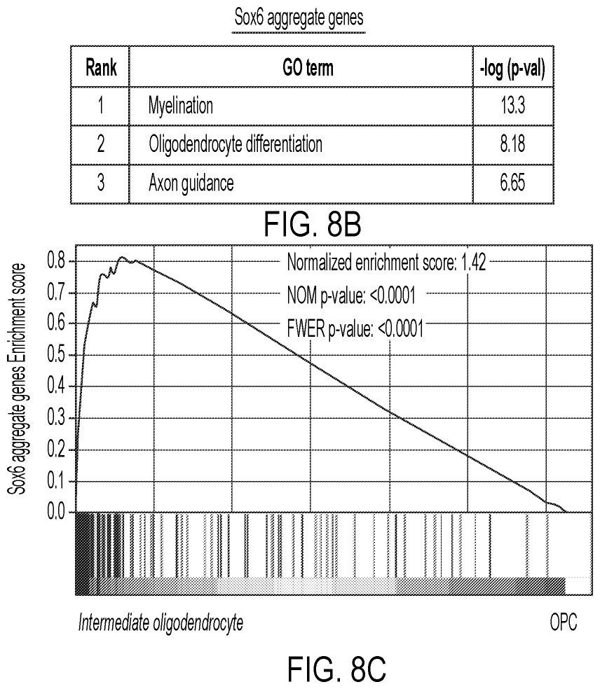

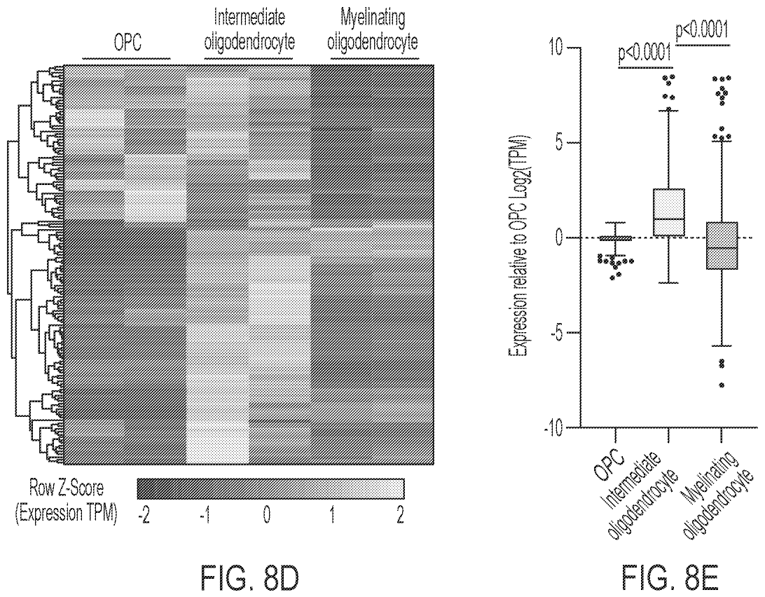

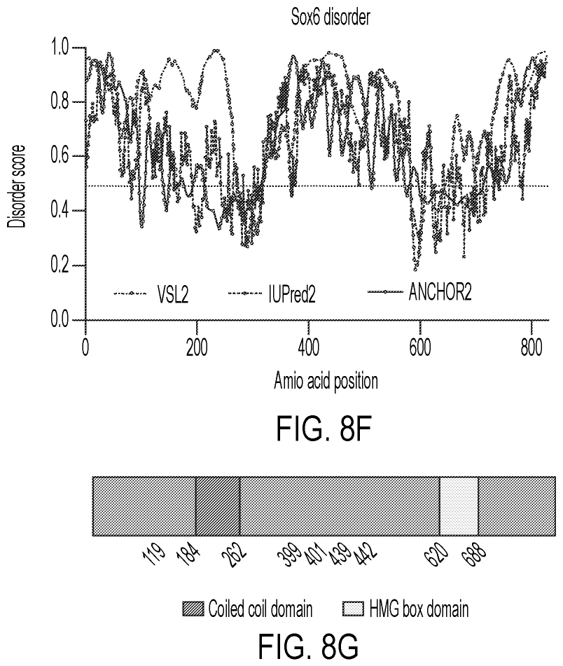

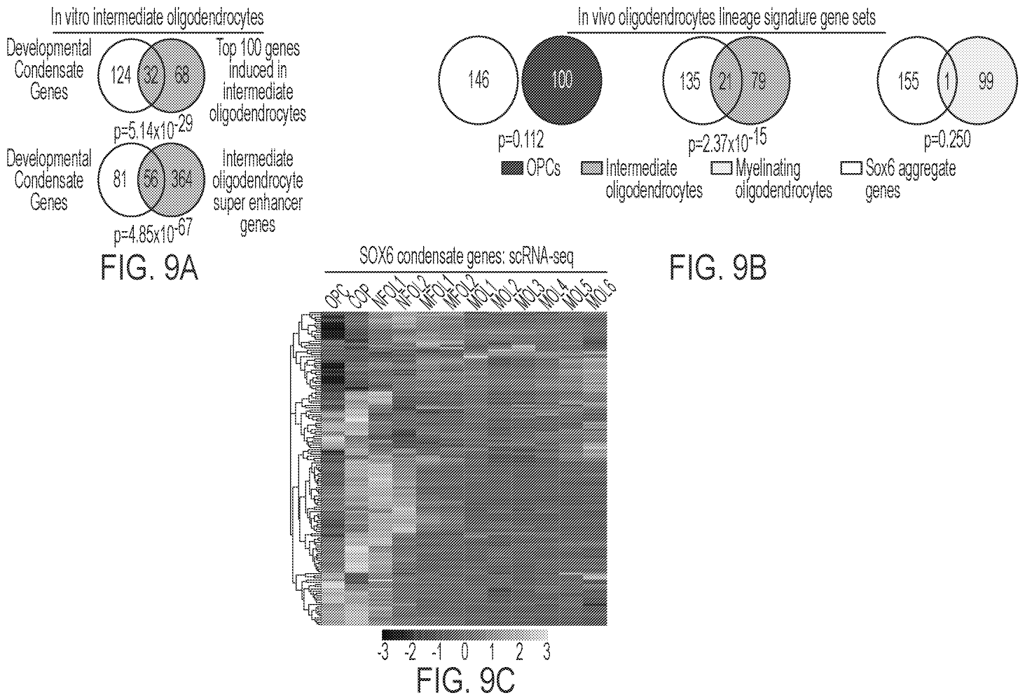

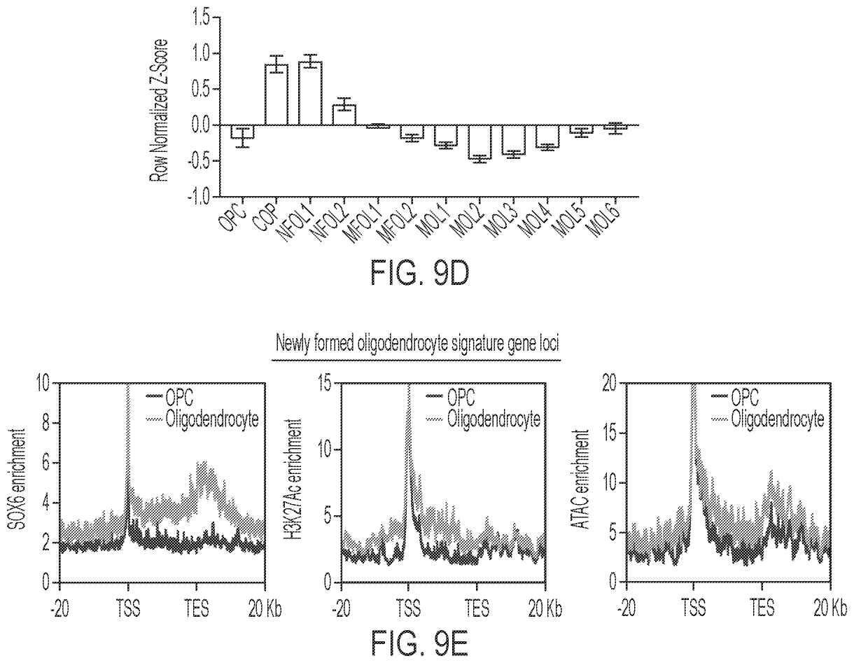

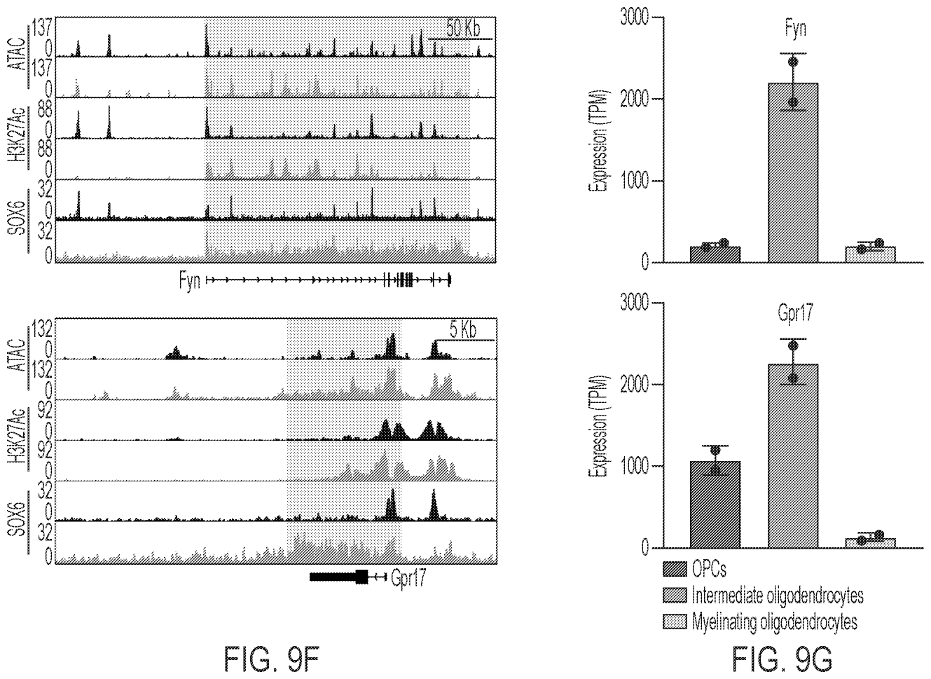

GO analysis of genes harboring SOX6 aggregation in intermediate oligodendrocytes, demonstrated a clear enrichment for oligodendrocyte differentiation pathways and were expressed most highly in intermediate oligodendrocytes compared to OPCs (FIGS. 8A-8C, and 9A). Specifically, these genes are strongly induced in intermediate oligodendrocytes and then decline during terminal differentiation to form fully mature myelinating oligodendrocytes (FIGS. 8D, 8E, and 9B-9G). Given the massive density of SOX6, an inherently disordered protein, at super-enhancer associated genes that increase sharply in intermediate oligodendrocytes and then decrease in myelinating oligodendrocytes, it was hypothesized that these regions represent SOX6 biomolecular condensates that stabilize, not inhibit, the intermediate oligodendrocyte state (FIGS. 8F and 8G) (Daneshvar et al., 2020; Henninger et al., 2020; Hnisz et al., 2017; Sabari et al., 2018). Performing high resolution confocal microscopy of immunofluorescence coupled with RNA-FISH confirmed Sox6 puncta in intermediate oligodendrocytes that co-localized with the predicted Sox6 aggregate gene Bcas1, whereas this co-localization was severely diminished at the non-aggregate gene Gapdh. Furthermore, these biomolecular condensates were dissolved upon addition of the drug JQ1, which has been demonstrated to dissolve nuclear transcriptional condensates.

Taken together, this data demonstrates that Sox6 forms nuclear condensates at genes transiently expressed along the trajectory of forming a fully mature myelinating oligodendrocyte.

### Example 7 Loss of Sox6 Impairs the Intermediate Oligodendrocyte State

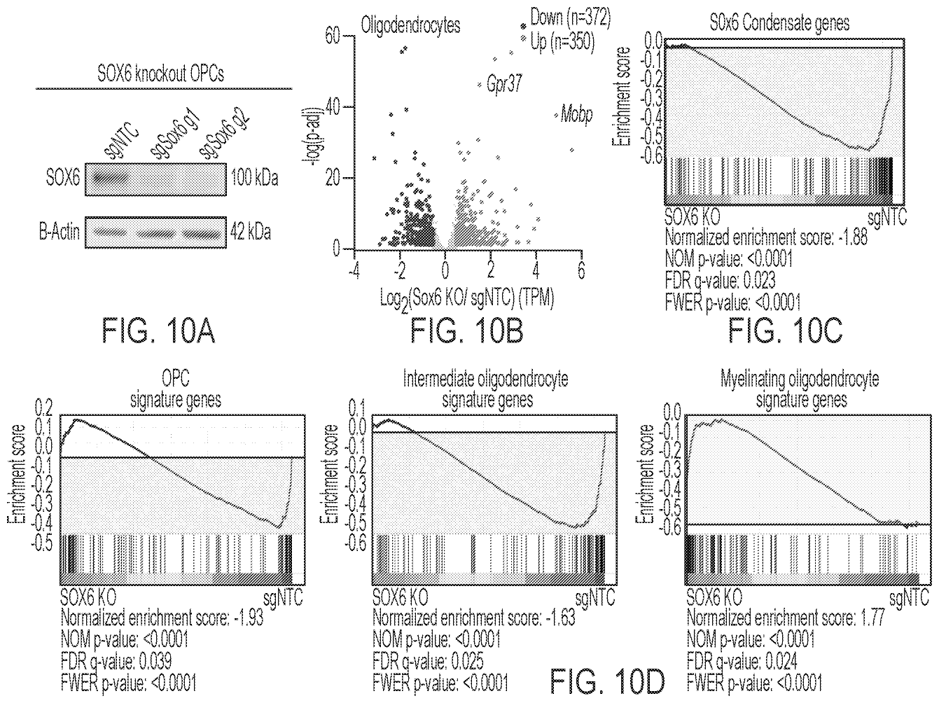

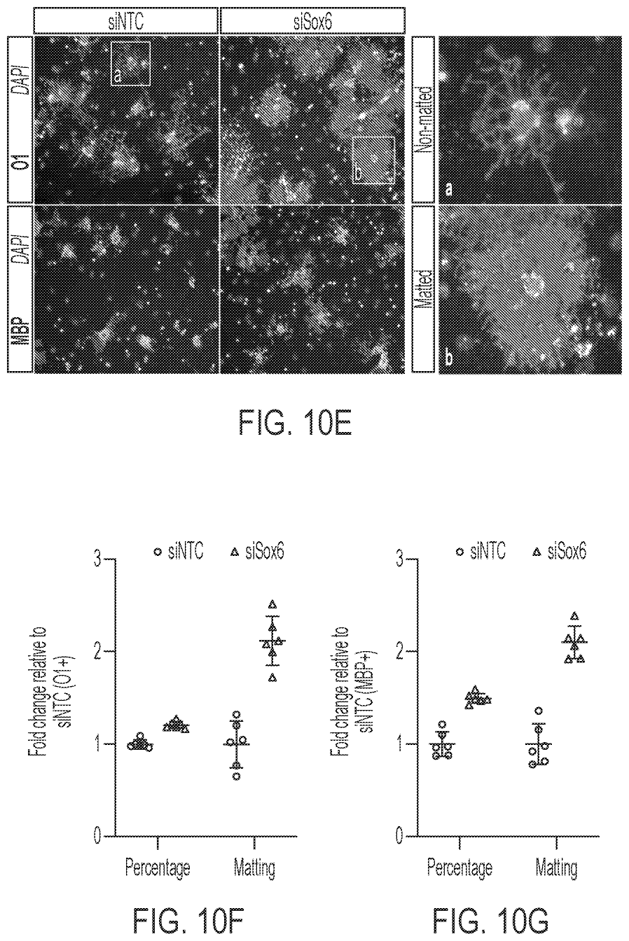

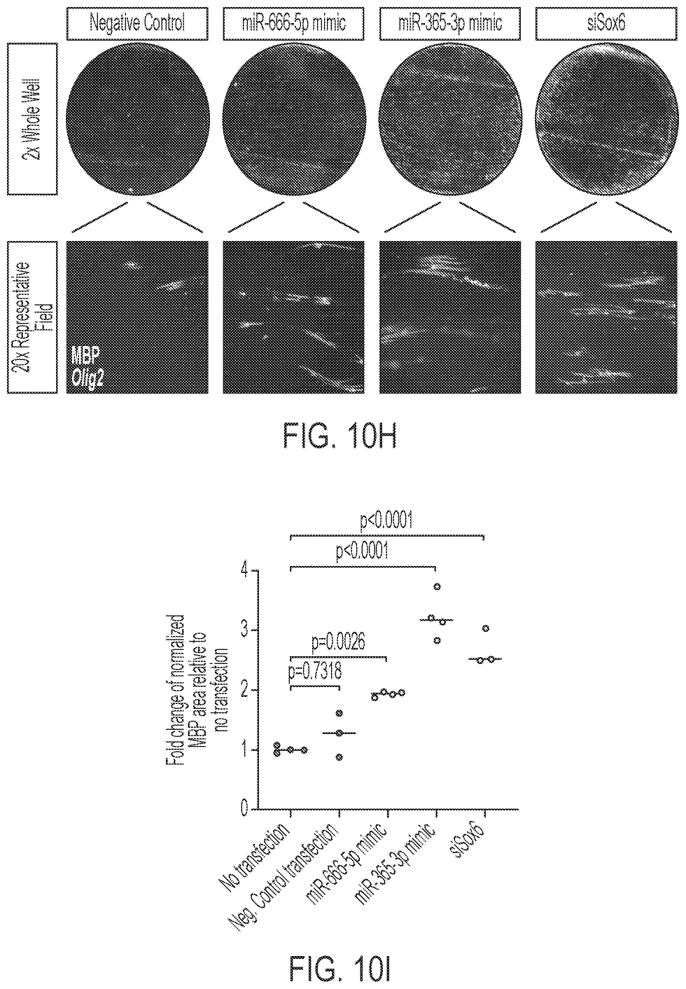

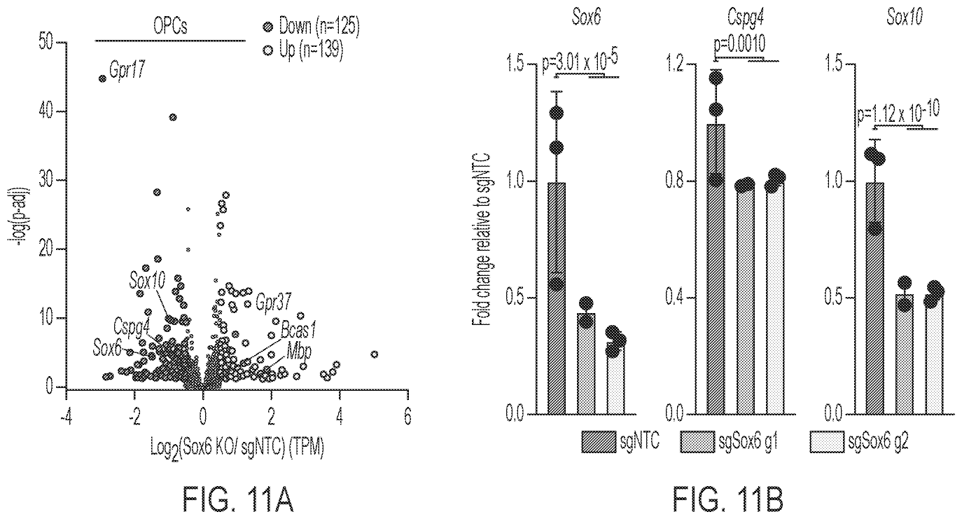

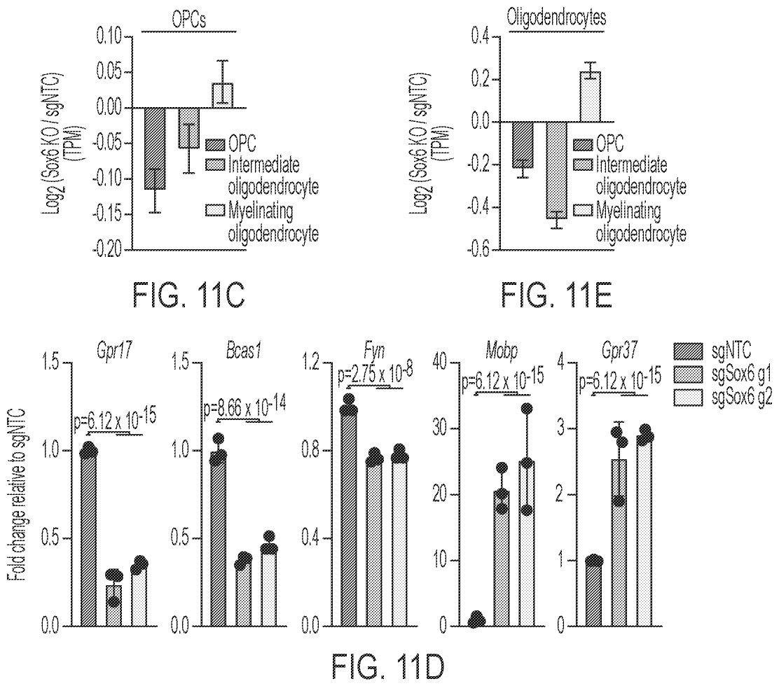

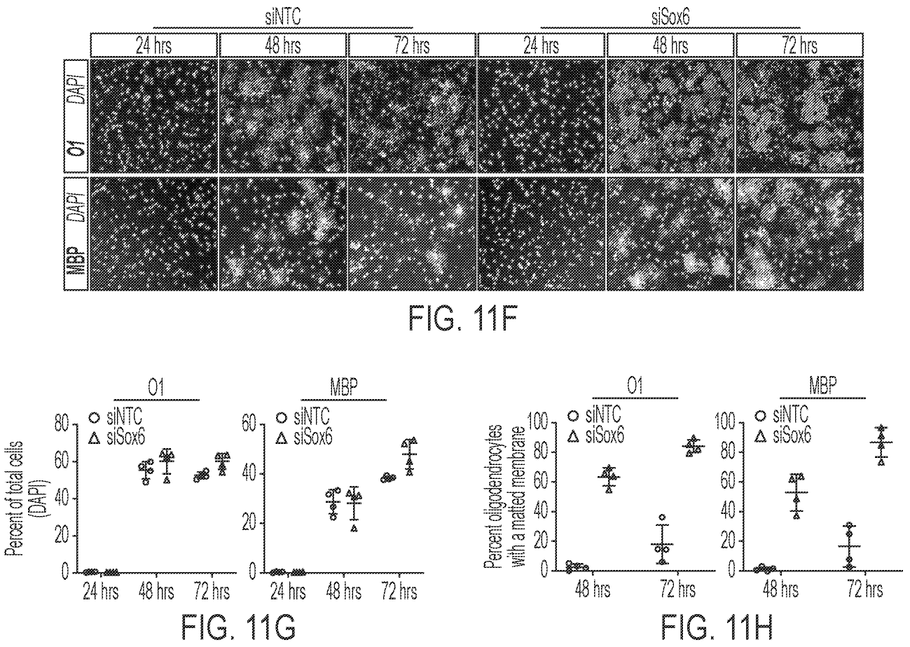

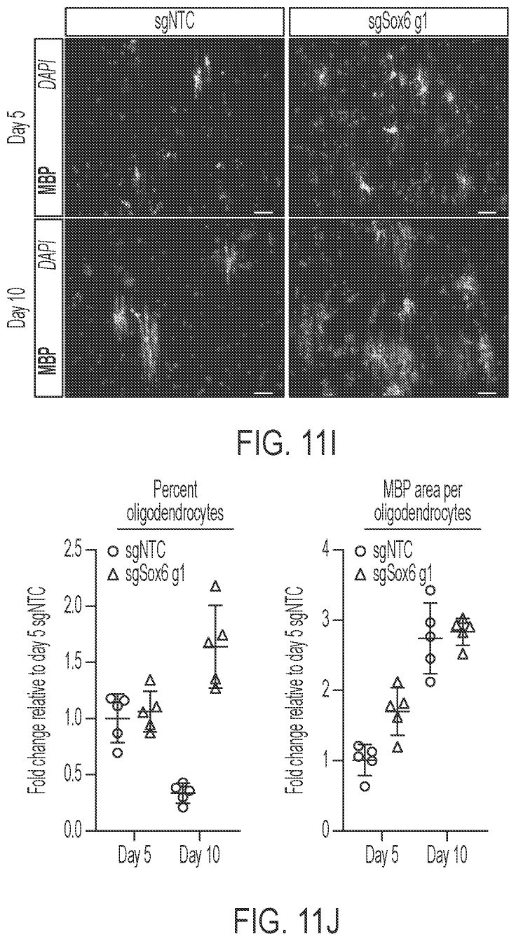

To test whether Sox6 plays a functional role at these condensates, SOX6 knockout OPCs were generated using CRISPR-Cas9 using two independent single guide RNAs (sgSox6 g1 and sgSox6 g2), which led to a strong reduction in SOX6 expression compared to non-targeting control (sgNTC) (FIGS. 10A and 11A). RNA-seq was then performed on SOX6 knockout OPCs and differentiating oligodendrocyte cultures compared to non-targeting controls (FIGS. 10B and 11A). In agreement with binding at nearly all super-enhancers in OPCs, Sox6 knockout OPCs harbored a reduction in OPC signature genes and transcription factors such as Cspg4 and Sox10 (FIGS. 11A-11C). In differentiating oligodendrocytes, loss of SOX6 led to a reduction in Sox6 condensate gene expression (FIGS. 10B, 10C, and 11D). In agreement with these condensate genes stabilizing genes transiently expressed by intermediate oligodendrocytes, SOX6 knockout oligodendrocytes harbored a significant reduction in intermediate oligodendrocyte gene expression (FIGS. 10D and 11E). However, SOX6 knockout OPCs exhibited a significant increase in mature myelinating oligodendrocyte signature genes, such as Mobp (FIGS. 10D, 11D, and 11E).

This data suggests that loss of SOX6 impairs the intermediate pre-myelinating oligodendrocyte state to skip directly to mature myelinating oligodendrocytes.

### Example 8 Loss of Sox6 Accelerates Oligodendrocyte Maturation

Given the pronounced acceleration of Sox6 knockout oligodendrocyte gene signatures, whether loss of SOX6 was driving a noticeable change in cell shape of oligodendrocytes formed in 2D culture was tested. As observed earlier, knockdown of Sox6 led to a slight increase in formation of O1 and MBP positive oligodendrocytes at day 3 of differentiation; however, the more dramatic finding was a stark appearance of oligodendrocytes harboring a complex and matted membrane with Sox6 knockdown, which agrees with their precocious maturity (FIGS. 10E-10G).

Performing a differentiation time course following Sox6 knockdown at days 1, 2, and 3 of differentiation demonstrated that this effect on membrane matting occurs concurrently with the appearance of O1 positive oligodendrocytes at day 2 of differentiation and once again is more pronounced than the increase in percentage of oligodendrocytes (FIGS. 11F-11H).

To further test this rapid induction of a myelinating oligodendrocyte signature, OPCs were transfected with miRNAs predicted to target Sox6, namely miR-666-5p and miR-365-3p, and siRNA targeting Sox6, which all led to a reduction in Sox6 expression and increased the formation of wrapping oligodendrocytes in a microfiber differentiation assay relative to control (Bechler et al., 2015) (FIGS. 10H, 10I, and 11I). Performing a time course of Sox6 knockout and control oligodendrocytes on microfibers demonstrated that loss of Sox6 accelerated the amount of MBP produced per oligodendrocyte with little effect on the percentage of oligodendrocytes at early time points (FIGS. 11J and 11K). At later time points, loss of Sox6 led to a dramatic increase in the number of oligodendrocytes compared to control, but the area of MBP per oligodendrocyte was identical to control.

Collectively, this data highlights a novel mechanism by which Sox6 governs the rate of oligodendrocyte maturation such that loss of Sox6 dramatically accelerates the formation of myelinating oligodendrocytes by destabilizing the intermediate oligodendrocyte state.

### Example 9 Sox6 Serves as a Master Regulator of Pre-Myelinating Oligodendrocytes by Maintaining Intermediate Cell State Through Stabilization of Transcriptional Condensates

Elucidating transcriptional regulatory networks provides clues as to how cells balance decisions from self-renewal to differentiation to responding to an environmental lesion and identification of dominant transcription factor nodes provides a roadmap that can be used to parse disease pathology (Lee and Young, 2013). For the oligodendrocyte lineage, a majority of what is known regarding core regulators of oligodendrocyte formation has relied on gene expression data and most studies are limited to studying the formation of fully mature myelinating oligodendrocytes and overlook the dominant regulators of intermediate pre-myelinating oligodendrocytes (Elbaz and Popko, 2019; Emery and Lu, 2015; He et al., 2016; Stolt et al., 2002; Sun et al., 2018).

Here, the unique access to scalable and pure populations of OPCs and pre-myelinating oligodendrocytes to rigorously interrogate core transcriptional networks utilizing diverse strategies was capitalized. Coupling super-enhancer profiling with functional miRNA screening pinpointed Sox6 as a highly connected and regulated node governing pre-myelinating oligodendrocyte formation (FIGS. 1A-1H, 4A-4F and 6A-6G). This pipeline also allowed for the discovery of miRNAs with potent impacts on oligodendrocyte formation that are not normally expressed in the oligodendrocyte lineage, and therefore would have been missed utilizing a gene expression approach.

The current understanding of Sox6 function in the oligodendrocyte lineage would lead us to predict that Sox6 should avoid oligodendrocyte super-enhancers during differentiation and that loss of Sox6 would increase the pre-myelinating oligodendrocyte gene signature (Baroti et al., 2016; Elbaz and Popko, 2019; Emery and Lu, 2015; Stolt et al., 2006). However, interrogation of the whole-genome binding profile of Sox6 in pre-myelinating oligodendrocytes revealed that Sox6 spread across the gene body at genes associated with super-enhancers (FIGS. 8A-8G). This pattern of Sox6 binding could represent the massive density of interacting proteins at super-enhancers, which lead to liquid-liquid phase separated droplets that drive transient high gene expression (Hnisz et al., 2017; Sabari et al., 2018; Shrinivas et al., 2019, Henninger et al, 2020). This suggested that Sox6 could be stabilizing transcriptional condensates at these genes, which is supported by the knockout studies demonstrating that loss of Sox6 effectively skips the intermediate oligodendrocyte state (FIGS. 10A-10J). This study demonstrates that dominant transcription factors could stabilize transcriptional condensates to maintain intermediate states within differentiation trajectories.

This work highlights Sox6 as a master regulator of pre-myelinating oligodendrocytes such that loss of Sox6 leads to precocious activation of a myelinating gene signature and skipping of the intermediate pre-myelinating state (FIGS. 10A-10I). This idea of separating differentiation from myelination has been demonstrated in select cases and suggests both processes have their own transcriptional networks (Gibson et al., 2019; Howng et al., 2010). Loss of Sox6 in OPCs has been shown to drive precocious myelin transcripts and proteins in vivo; however, myelin formation in white matter tracts is dramatically impaired and leads to premature death of knockout mice (Baroti et al., 2016). This could suggest that precocious myelinating oligodendrocytes formed in the context of Sox6 knockout are functionally impaired and stresses the potential importance of proceeding through the intermediate oligodendrocyte state. This would have profound implications for ensuring proper functionality of differentiated cells generated by direct reprogramming to be used in regenerative medicine (Xu et al., 2015). It is also possible that the precocious myelinating oligodendrocytes are functional but impaired migration of Sox6 knockout OPCs prevents formation of oligodendrocytes in proper locations within white matter tracts (Baroti et al., 2016). If this is the case, targeting Sox6 could offer a therapeutic approach in the context of numerous myelin diseases, such as Multiple Sclerosis, in which OPCs are documented to proliferate and migrate to lesioned areas but are unable to form mature myelinating oligodendrocytes (Gibson et al., 2019; Kuhlmann et al., 2008; Mathys et al., 2019; Wang et al., 2020; Yeung et al., 2019).

Collectively, this work dissects the transcriptional networks governing pre-myelinating oligodendrocyte formation and establishes a novel role for Sox6 maintaining this intermediate cell state through stabilization of transcriptional condensates. More broadly, this work establishes a novel mechanism by which transcription factors stabilize distinct intermediate stages along the continuum of differentiation.

### Example 10 Post-Natal Inactivation of Sox6 Using AAV Delivery of CRISPR/CAS9

The data presented herein demonstrates that inhibition of Sox6 function promotes maturation of OPCs to ODC. This example demonstrates that post-natal inactivation of Sox6 can be used to effectively treat or at least reduce the severity of diseases associated with loss or reduction of oligodendrocyte (ODC) function, by enhancing oligodendrocyte maturation and/or functional myelin production, n order to treat patients (including children and adults) having myelin diseases, such as those characterized by myelin damage or loss (e.g., Pelizaeus Merzbacher Disease (PMD)).

To facilitate the delivery of the CRISPR/Cas9 system with sgRNA targeting the Sox6 locus in a patient, several CNS-targeted AAV serotypes, including PHP.B, are generated, and AAV tropism for OPCs is validated. The AAV constructs (AAV9 or AAV-PHP.B) contain a SaCRISPR-Cas9 nuclease (CMV-SaCas9, SaCas9 coding sequence under the control of a CMV promoter) with a site directed guide RNA (sgRNA) against Sox6 (U6-sgRNA, the sgRNA is under the control of the U6 promotor), which is designed to generate indels in the Sox6 gene and thus prevent expression of a functional Sox6 protein.

Mice are maintained in accordance with approved protocols reviewed by Case Western Reserve University's Institutional Animal Care and Use Committee. Mice are housed in a temperature and humidity controlled housing unit under a 12 hour day/light cycle and are allowed ad libitum access to food.

Experimental animals (e.g., mice, such as Jimpy—a severe mouse model of Pelizaeus Merzbacher Disease) are then treated via stereotaxic intraventricular injection with the CRISPR-containing AAVs or controls encoding GFP. Specifically, male postnatal day 0 pups were obtained from (jimpy) mice breeding pairs and rapidly anesthetized using cryoanesthesia.

A 10 μL Hamilton syringe with a 32 gauge needle is loaded with AAV (AAV9 or AAV-PHP.B) packaging CMV-SaCas9 and U6-sgRNA targeting Sox 6. The needle is lowered through the skull to a depth of about 2 mm at a position 2/5 from the intersection of the sagittal suture and lambdoid to the eye. 2 μL of viral solution was injected into the lateral ventricle. The injection is then repeated in the contralateral lateral ventricle using the same coordinates, and injection volume for a total delivery of about 1×10¹⁰-1×10¹¹ vector genomes to the ventricular system. The same is repeated for control mice.

Pups are allowed to recover on a heating pad and then reintroduced to their mother. Pups are monitored daily for phenotype improvement as compared to untreated or vehicle-treated animals, which develop severe (motor) phenotype (e.g., intention tremor and seizures) by 2 weeks of age and death by 3 weeks of age.

Treated animals surviving beyond 3 weeks are analyzed using behavioral (e.g., rotarod and open field testing for motor performance, histology (immuno staining of the CNS for myelin proteins and electron microscopy for myelin ultrastructure), or daily monitoring for lifespan extension statistical analysis.

Further, the spatial requirements for the number of cells required to be edited in the CNS (“dose response” of gene edited cells) are determined to generate a functional response with immunohistochemistry.

Finally, the temporal relationship is explored by introducing an optimized construct at post-natal days P1, P7 and P14. An optimized CRISPR/Cas9 approach within a defined therapeutic window reduces expression of the Sox6 protein, increases lifespan of treated individual, and restores myelination of axons.

### Example 11 Post-Natal Knockdown of Sox6 Using Antisense Oligonucleotides (ASO)

This example is carried out to demonstrate that post-natal down-regulation or knockdown of Sox6 gene activity using ASO can be used to effectively treat or reduce severity of a myelin disease, such as PMD.

Mice are maintained in accordance with approved protocols reviewed by Case Western Reserve University's Institutional Animal Care and Use Committee. Mice are housed in a temperature and humidity controlled housing unit under a 12 hour day/light cycle and are allowed ad libitum access to food.

Male postnatal day 0 pups are obtained from jimpy (a severe mouse model of Pelizaeus Merzbacher Disease) breeding pairs and rapidly anesthetized using cryoanesthesia. A 10 pL Hamilton syringe with a 32 gauge needle is loaded with antisense oligonucleotides targeting Sox6. The needle is lowered through the skull to a depth of about 2 mm at a position 2/5 from the intersection of the sagittal suture and lambdoid to the eye. 2 pL of ASO solution is injected into the lateral ventricle. The injection is then repeated in the contralateral lateral ventricle using the same coordinates and injection volume for a total delivery of about 10-75 pg of ASO to the ventricular system.

Pups are allowed to recover on a heating pad and then reintroduced to their mother. Pups are monitored daily for phenotype improvement as compared to untreated or vehicle-treated jimpy animals, which develop severe motor phenotype (e.g., intention tremor and seizures) by 2 weeks of age and death by 3 weeks of age.

Treated animals surviving beyond 3 weeks are analyzed using behavioral (e.g., rotarod and open field testing for motor performance), histology (immuno staining of the CNS for myelin proteins and electron microscopy for myelin ultrastructure), or daily monitoring for lifespan extension statistical analysis.

### Example 12 Post-Natal Knockdown of Sox6 Using RNAi

This example is carried out to demonstrate that post-natal down-regulation or knockdown of Sox6 gene activity using RNAi can be used to effectively treat or reduce severity of a myelin disease, such as PMD.

Mice are maintained in accordance with approved protocols reviewed by Case Western Reserve University's Institutional Animal Care and Use Committee. Mice are housed in a temperature and humidity controlled housing unit under a 12 hour day/light cycle and are allowed ad libitum access to food.

Male postnatal day 0 pups are obtained from jimpy (a severe mouse model of Pelizaeus Merzbacher Disease) breeding pairs and rapidly anesthetized using cryoanesthesia. A 10 pL Hamilton syringe with a 32 gauge needle is loaded with AAV (AAV9 or AAV-PHP.B) packaging CMV-RNAi targeting Sox6 (an RNAi construct that can be transcribed inside the cell under the control of a CMV promotor to generate a functional RNAi molecules, which can then be processed to siRNA/shRNA/miRNA targeting Sox6).

Alternatively, the needle is loaded with a non-viral formulation of the RNAi construct targeting Sox6, using non-viral siRNA carriers such as cell-penetrating peptides, polymers, dendrimers, siRNA bioconjugates, and lipid-based siRNA carriers, etc. The needle is lowered through the skull to a depth of about 2 mm at a position 2/5 from the intersection of the sagittal suture and lambdoid to the eye. 2 pL of RNAi solution is injected into the lateral ventricle. The injection is then repeated in the contralateral lateral ventricle using the same coordinates and injection volume.

Pups are allowed to recover on a heating pad and then reintroduced to their mother. Pups are monitored daily for phenotype improvement as compared to untreated or vehicle-treated jimpy animals, which develop severe motor phenotype (e.g, intention tremor and seizures) by 2 weeks of age and death by 3 weeks of age.

Treated animals surviving beyond 3 weeks are analyzed using behavioral (e.g., rotarod and open field testing for motor performance), histology (immuno staining of the CNS for myelin proteins and electron microscopy for myelin ultrastructure), or daily monitoring for lifespan extension statistical analysis.

### REFERENCES

Adam, R. C., Yang, H., Rockowitz, S., Larsen, S. B., Nikolova, M., Oristian, D. S., Polak, L., Kadaja, M., Asare, A., Zheng, D., et al. (2015). Pioneer factors govern super-enhancer dynamics in stem cell plasticity and lineage choice. Nature 521, 366-370.
Allan, K. C., Hu, L. R., Scavuzzo, M. A., Morton, A. R., Gevorgyan, A. S., Cohn, E. F., Clayton, B. L. L., Bederman, I. R., Hung, S., Bartels, C. F., et al. (2020). Non-canonical Targets of HIF1a Impair Oligodendrocyte Progenitor Cell Function. Cell Stem Cell.
Ampofo, E., Schmitt, B. M., Menger, M. D., and Laschke, M. W. (2017). The regulatory mechanisms of NG2/CSPG4 expression. Cell Mol Biol Lett 22, 4.
Baroti, T., Zimmermann, Y., Schillinger, A., Liu, L., Lommes, P., Wegner, M., and Stolt, C. C. (2016). Transcription factors Sox5 and Sox6 exert direct and indirect influences on oligodendroglial migration in spinal cord and forebrain. Glia 64, 122-138.
Bechler, M. E., Byrne, L., and Ffrench-Constant, C. (2015). CNS Myelin Sheath Lengths Are an Intrinsic Property of Oligodendrocytes. Curr Biol 25, 2411-2416.
Bleu, M., Gaulis, S., Lopes, R., Sprouffske, K., Apfel, V., Holwerda, S., Pregnolato, M., Yildiz, U., Cordo, V., Dost, A. F. M., et al. (2019). PAX8 activates metabolic genes via enhancer elements in Renal Cell Carcinoma. Nat Commun 10, 3739.
Boyer, L. A., Lee, T. I., Cole, M. F., Johnstone, S. E., Levine, S. S., Zucker, J. P., Guenther, M. G., Kumar, R. M., Murray, H. L., Jenner, R. G., et al. (2005). Core transcriptional regulatory circuitry in human embryonic stem cells. Cell 122, 947-956.
Brown, J. D., Lin, C. Y., Duan, Q., Griffin, G., Federation, A., Paranal, R. M., Bair, S., Newton, G., Lichtman, A., Kung, A., et al. (2014). NF-kappaB directs dynamic super enhancer formation in inflammation and atherogenesis. Mol Cell 56, 219-231.
Chen, Y., Wu, H., Wang, S., Koito, H., Li, J., Ye, F., Hoang, J., Escobar, S. S., Gow, A., Arnett, H. A., et al. (2009). The oligodendrocyte-specific G protein-coupled receptor GPR17 is a cell-intrinsic timer of myelination. Nat Neurosci 12, 1398-1406.
Chipumuro, E., Marco, E., Christensen, C. L., Kwiatkowski, N., Zhang, T., Hatheway, C. M., Abraham, B. J., Sharma, B., Yeung, C., Altabef, A., et al. (2014). CDK7 inhibition suppresses super-enhancer-linked oncogenic transcription in MYCN-driven cancer. Cell 159, 1126-1139.
Corces, M. R., Trevino, A. E., Hamilton, E. G., Greenside, P. G., Sinnott-Armstrong, N. A., Vesuna, S., Satpathy, A. T., Rubin, A. J., Montine, K. S., Wu, B., et al. (2017). An improved ATAC-seq protocol reduces background and enables interrogation of frozen tissues. Nature methods 14, 959-962.
Creyghton, M. P., Cheng, A. W., Welstead, G. G., Kooistra, T., Carey, B. W., Steine, E. J., Hanna, J., Lodato, M. A., Frampton, G. M., Sharp, P. A., et al. (2010). Histone H3K27ac separates active from poised enhancers and predicts developmental state. Proc Natl Acad Sci USA 107, 21931-21936.
Daneshvar, K., Ardehali, M. B., Klein, I. A., Hsieh, F. K., Kratkiewicz, A. J., Mahpour, A., Cancelliere, S. O. L., Zhou, C., Cook, B. M., Li, W., et al. (2020). lncRNA DIGIT and BRD3 protein form phase-separated condensates to regulate endoderm differentiation. Nat Cell Biol 22, 1211-1222.
Doench, J. G., Fusi, N., Sullender, M., Hegde, M., Vaimberg, E. W., Donovan, K. F., Smith, I., Tothova, Z., Wilen, C., Orchard, R., et al. (2016). Optimized sgRNA design to maximize activity and minimize off-target effects of CRISPR-Cas9. Nat Biotechnol 34, 184-191.
Duan, Q., Mao, X., Xiao, Y., Liu, Z., Wang, Y., Zhou, H., Zhou, Z., Cai, J., Xia, K., Zhu, Q., et al. (2016). Super enhancers at the miR-146a and miR-155 genes contribute to self-regulation of inflammation. Biochim Biophys Acta 1859, 564-571.
Dugas, J. C., Cuellar, T. L., Scholze, A., Ason, B., Ibrahim, A., Emery, B., Zamanian, J. L., Foo, L. C., McManus, M. T., and Barres, B. A. (2010). Dicer1 and miR-219 Are required for normal oligodendrocyte differentiation and myelination. Neuron 65, 597-611.
Elbaz, B., and Popko, B. (2019). Molecular Control of Oligodendrocyte Development. Trends Neurosci 42, 263-277.
Elitt, M. S., Shick, H. E., Madhavan, M., Allan, K. C., Clayton, B. L. L., Weng, C., Miller, T. E., Factor, D. C., Barbar, L., Nawash, B. S., et al. (2018). Chemical Screening Identifies Enhancers of Mutant Oligodendrocyte Survival and Unmasks a Distinct Pathological Phase in Pelizaeus-Merzbacher Disease. Stem Cell Reports 11, 711-726.
Emery, B. (2010). Regulation of oligodendrocyte differentiation and myelination. Science 330, 779-782.
Emery, B., and Lu, Q. R. (2015). Transcriptional and Epigenetic Regulation of Oligodendrocyte Development and Myelination in the Central Nervous System. Cold Spring Harb Perspect Biol 7, a020461.
Enver, T., Pera, M., Peterson, C., and Andrews, P. W. (2009). Stem cell states, fates, and the rules of attraction. Cell Stem Cell 4, 387-397.
Factor, D. C., Barbeau, A. M., Allan, K. C., Hu, L. R., Madhavan, M., Hoang, A. T., Hazel, K. E. A., Hall, P. A., Nisraiyya, S., Najm, F. J., et al. (2020). Cell Type-Specific Intralocus Interactions Reveal Oligodendrocyte Mechanisms in MS. Cell 181, 382-395 e321.
Federation, A. J. P., D. R.; Ott, C. J.; Fan, A.; Lin, C. Y.; Bradner, J. E. (2018). Identification of candidate master transcription factors within enhancer-centric transcriptional regulatory networks. bioRxiv.
Friard, O., Re, A., Taverna, D., De Bortoli, M., and Cora, D. (2010). CircuitsDB: a database of mixed microRNA/transcription factor feed-forward regulatory circuits in human and mouse. BMC Bioinformatics 11, 435.
Gao, F. B., Apperly, J., and Raff, M. (1998). Cell-intrinsic timers and thyroid hormone regulate the probability of cell-cycle withdrawal and differentiation of oligodendrocyte precursor cells. Dev Biol 197, 54-66.
Gibson, E. M., Nagaraja, S., Ocampo, A., Tam, L. T., Wood, L. S., Pallegar, P. N., Greene, J. J., Geraghty, A. C., Goldstein, A. K., Ni, L., et al. (2019). Methotrexate Chemotherapy Induces Persistent Tri-glial Dysregulation that Underlies Chemotherapy-Related Cognitive Impairment. Cell 176, 43-55 e13.
Gryder, B. E., Pomella, S., Sayers, C., Wu, X. S., Song, Y., Chiarella, A. M., Bagchi, S., Chou, H. C., Sinniah, R. S., Walton, A., et al. (2019a). Histone hyperacetylation disrupts core gene regulatory architecture in rhabdomyosarcoma. Nat Genet 51, 1714-1722.
Gryder, B. E., Wu, L., Woldemichael, G. M., Pomella, S., Quinn, T. R., Park, P. M. C., Cleveland, A., Stanton, B. Z., Song, Y., Rota, R., et al. (2019b). Chemical genomics reveals histone deacetylases are required for core regulatory transcription. Nat Commun 10, 3004.
Gryder, B. E., Yohe, M. E., Chou, H. C., Zhang, X., Marques, J., Wachtel, M., Schaefer, B., Sen, N., Song, Y., Gualtieri, A., et al. (2017). PAX3-FOXO1 Establishes Myogenic Super Enhancers and Confers BET Bromodomain Vulnerability. Cancer Discov 7, 884-899.
He, D., Marie, C., Zhao, C., Kim, B., Wang, J., Deng, Y., Clavairoly, A., Frah, M., Wang, H., He, X., et al. (2016). Chd7 cooperates with Sox10 and regulates the onset of CNS myelination and remyelination. Nat Neurosci 19, 678-689.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Henninger, J. E., Oksuz, O., Shrinivas, K., Sagi, I., LeRoy, G., Zheng, M. M., Andrews, J. O., Zamudio, A. V., Lazaris, C., Hannett, N. M., et al. (2020). RNA-Mediated Feedback Control of Transcriptional Condensates. Cell.
Hnisz, D., Abraham, B. J., Lee, T. I., Lau, A., Saint-Andre, V., Sigova, A. A., Hoke, H. A., and Young, R. A. (2013). Super-enhancers in the control of cell identity and disease. Cell 155, 934-947.
Hnisz, D., Shrinivas, K., Young, R. A., Chakraborty, A. K., and Sharp, P. A. (2017). A Phase Separation Model for Transcriptional Control. Cell 169, 13-23.
Howng, S. Y., Avila, R. L., Emery, B., Traka, M., Lin, W., Watkins, T., Cook, S., Bronson, R., Davisson, M., Barres, B. A., et al. (2010). ZFP191 is required by oligodendrocytes for CNS myelination. Genes Dev 24, 301-311.
Hubler, Z., Allimuthu, D., Bederman, I., Elitt, M. S., Madhavan, M., Allan, K. C., Shick, H. E., Garrison, E., M, T. K., Factor, D. C., et al. (2018). Accumulation of 8,9-unsaturated sterols drives oligodendrocyte formation and remyelination. Nature 560, 372-376.
Iacono, G., Massoni-Badosa, R., and Heyn, H. (2019). Single-cell transcriptomics unveils gene regulatory network plasticity. Genome Biol 20, 110.
Kuhlmann, T., Miron, V., Cui, Q., Wegner, C., Antel, J., and Bruck, W. (2008). Differentiation block of oligodendroglial progenitor cells as a cause for remyelination failure in chronic multiple sclerosis. Brain 131, 1749-1758.
Lee, T. I., and Young, R. A. (2013). Transcriptional regulation and its misregulation in disease. Cell 152, 1237-1251.
Loven, J., Hoke, H. A., Lin, C. Y., Lau, A., Orlando, D. A., Vakoc, C. R., Bradner,
J. E., Lee, T. I., and Young, R. A. (2013). Selective inhibition of tumor oncogenes by disruption of super-enhancers. Cell 153, 320-334.
MacLean, A. L., Hong, T., and Nie, Q. (2018). Exploring intermediate cell states through the lens of single cells. Curr Opin Syst Biol 9, 32-41.
Marson, A., Levine, S. S., Cole, M. F., Frampton, G. M., Brambrink, T., Johnstone, S., Guenther, M. G., Johnston, W. K., Wernig, M., Newman, J., et al. (2008). Connecting microRNA genes to the core transcriptional regulatory circuitry of embryonic stem cells. Cell 134, 521-533.
Mathys, H., Davila-Velderrain, J., Peng, Z., Gao, F., Mohammadi, S., Young, J. Z., Menon, M., He, L., Abdurrob, F., Jiang, X., et al. (2019). Single-cell transcriptomic analysis of Alzheimer's disease. Nature 570, 332-337.
Mercado, N., Schutzius, G., Kolter, C., Estoppey, D., Bergling, S., Roma, G., Gubser Keller, C., Nigsch, F., Salathe, A., Terranova, R., et al. (2019). IRF2 is a master regulator of human keratinocyte stem cell fate. Nat Commun 10, 4676.
Monje, M. (2018). Myelin Plasticity and Nervous System Function. Annu Rev Neurosci 41, 61-76.
Montague, P., McCallion, A. S., Davies, R. W., and Griffiths, I. R. (2006). Myelin-associated oligodendrocytic basic protein: a family of abundant CNS myelin proteins in search of a function. Dev Neurosci 28, 479-487.
Mullen, A. C., Orlando, D. A., Newman, J. J., Loven, J., Kumar, R. M., Bilodeau, S., Reddy, J., Guenther, M. G., DeKoter, R. P., and Young, R. A. (2011). Master transcription factors determine cell-type-specific responses to TGF-beta signaling. Cell 147, 565-576.
Najm, F. J., Madhavan, M., Zaremba, A., Shick, E., Karl, R. T., Factor, D. C., Miller, T. E., Nevin, Z. S., Kantor, C., Sargent, A., et al. (2015). Drug-based modulation of endogenous stem cells promotes functional remyelination in vivo. Nature 522, 216-220.
Najm, F. J., Zaremba, A., Caprariello, A. V., Nayak, S., Freundt, E. C., Scacheri, P. C., Miller, R. H., and Tesar, P. J. (2011). Rapid and robust generation of functional oligodendrocyte progenitor cells from epiblast stem cells. Nature methods 8, 957-962.
Nave, K. A. (2010). Myelination and the trophic support of long axons. Nat Rev Neurosci 11, 275-283.
Ott, C. J., Federation, A. J., Schwartz, L. S., Kasar, S., Klitgaard, J. L., Lenci, R., Li, Q., Lawlor, M., Fernandes, S. M., Souza, A., et al. (2018). Enhancer Architecture and Essential Core Regulatory Circuitry of Chronic Lymphocytic Leukemia. Cancer Cell 34, 982-995 e987.
Sabari, B. R., Dall'Agnese, A., Boija, A., Klein, I. A., Coffey, E. L., Shrinivas, K., Abraham, B. J., Hannett, N. M., Zamudio, A. V., Manteiga, J. C., et al. (2018). Coactivator condensation at super-enhancers links phase separation and gene control. Science 361.
Sanjana, N. E., Shalem, O., and Zhang, F. (2014). Improved vectors and genome-wide libraries for CRISPR screening. Nature methods 11, 783-784.
Shaulian, E., and Karin, M. (2001). AP-1 in cell proliferation and survival. Oncogene 20, 2390-2400.
Shrinivas, K., Sabari, B. R., Coffey, E. L., Klein, I. A., Boija, A., Zamudio, A. V., Schuijers, J., Hannett, N. M., Sharp, P. A., Young, R. A., et al. (2019). Enhancer Features that Drive Formation of Transcriptional Condensates. Mol Cell 75, 549-561 e547.
Stolt, C. C., Rehberg, S., Ader, M., Lommes, P., Riethmacher, D., Schachner, M., Bartsch, U., and Wegner, M. (2002). Terminal differentiation of myelin-forming oligodendrocytes depends on the transcription factor Sox10. Genes Dev 16, 165-170.
Stolt, C. C., Schlierf, A., Lommes, P., Hillgartner, S., Werner, T., Kosian, T., Sock, E., Kessaris, N., Richardson, W. D., Lefebvre, V., et al. (2006). SoxD proteins influence multiple stages of oligodendrocyte development and modulate SoxE protein function. Dev Cell 11, 697-709.
Sun, L. O., Mulinyawe, S. B., Collins, H. Y., Ibrahim, A., Li, Q., Simon, D. J., Tessier-Lavigne, M., and Barres, B. A. (2018). Spatiotemporal Control of CNS Myelination by Oligodendrocyte Programmed Cell Death through the TFEB-PUMA Axis. Cell 175, 1811-1826 e1821.
Suzuki, H. I., Young, R. A., and Sharp, P. A. (2017). Super-Enhancer-Mediated RNA Processing Revealed by Integrative MicroRNA Network Analysis. Cell 168, 1000-1014 e1015.
Tiscornia, G., and Izpisua Belmonte, J. C. (2010). MicroRNAs in embryonic stem cell function and fate. Genes Dev 24, 2732-2741.
Tripathi, A., Volsko, C., Garcia, J. P., Agirre, E., Allan, K. C., Tesar, P. J., Trapp, B. D., Castelo-Branco, G., Sim, F. J., and Dutta, R. (2019). Oligodendrocyte Intrinsic miR-27a Controls Myelination and Remyelination. Cell Rep 29, 904-919 e909.
Tsankov, A. M., Gu, H., Akopian, V., Ziller, M. J., Donaghey, J., Amit, I., Gnirke, A., and Meissner, A. (2015). Transcription factor binding dynamics during human ES cell differentiation. Nature 518, 344-349.
Vlachos, I. S., Zagganas, K., Paraskevopoulou, M. D., Georgakilas, G., Karagkouni, D., Vergoulis, T., Dalamagas, T., and Hatzigeorgiou, A. G. (2015). DIANA-miRPath v3.0: deciphering microRNA function with experimental support. Nucleic Acids Res 43, W460-466.
Wang, J., He, X., Meng, H., Li, Y., Dmitriev, P., Tian, F., Page, J. C., Lu, Q. R., and He, Z. (2020). Robust Myelination of Regenerated Axons Induced by Combined Manipulations of GPR17 and Microglia. Neuron 108, 876-886 e874.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Xu, J., Du, Y., and Deng, H. (2015). Direct lineage reprogramming: strategies, mechanisms, and applications. Cell Stem Cell 16, 119-134.
Yang, H. J., Vainshtein, A., Maik-Rachline, G., and Peles, E. (2016). G protein-coupled receptor 37 is a negative regulator of oligodendrocyte differentiation and myelination. Nat Commun 7, 10884.
Yeung, M. S. Y., Djelloul, M., Steiner, E., Bernard, S., Salehpour, M., Possnert, G., Brundin, L., and Frisen, J. (2019). Dynamics of oligodendrocyte generation in multiple sclerosis. Nature 566, 538-542.
Zhang, Y., Chen, K., Sloan, S. A., Bennett, M. L., Scholze, A. R., O'Keeffe, S., Phatnani, H. P., Guarnieri, P., Caneda, C., Ruderisch, N., et al. (2014). An RNA-sequencing transcriptome and splicing database of glia, neurons, and vascular cells of the cerebral cortex. J Neurosci 34, 11929-11947.
Zhao, X., He, X., Han, X., Yu, Y., Ye, F., Chen, Y., Hoang, T., Xu, X., Mi, Q. S., Xin, M., et al. (2010). MicroRNA-mediated control of oligodendrocyte differentiation. Neuron 65, 612-626.
Zhou, Q., Choi, G., and Anderson, D. J. (2001). The bHLH transcription factor Olig2 promotes oligodendrocyte differentiation in collaboration with Nkx2.2. Neuron 31, 791-807.
Zhou, Y., Zhou, B., Pache, L., Chang, M., Khodabakhshi, A. H., Tanaseichuk, O., Benner, C., and Chanda, S. K. (2019). Metascape provides a biologist-oriented resource for the analysis of systems-level datasets. Nat Commun 10, 1523.

1. A method for accelerating cellular maturation of a cell, the method comprising impairing the activity of developmental transcriptional condensates at an intermediate stage of a cell lineage for the cell.

2. The method of claim 1, wherein the cell is an oligodendrocyte.

3. The method of claim 1 or 2, wherein the developmental transcriptional condensates are regulated by Sox6.

4. The method of claim 3, wherein the method comprises delivering to the cell an agent to decrease expression of endogenous Sox6.

5. The method of claim 4, wherein the agent is an antisense oligonucleotide (ASO), an siRNA, a CRISPR interference agent (Cas effector enzyme and guide RNA), TALE/zinc finger protein, NgAO, micro RNA, or a coding sequence therefor.

6. The method of any one of claims 4-5, wherein the method comprises contacting the cell with a delivery vehicle comprising the agent or the coding sequence therefor.

7. The method of any one of claims 4-6, wherein the delivery vehicle is an AAV vector, an adenoviral vector, or a lentivirus vector.

8. The method of any one of claims 6-7, wherein the cell is contacted in vitro, in vivo, or ex vivo.

9. The method of any one of claims 1-8, wherein the method accelerates oligodendrocyte maturation.

10. The method of any one of claims 1-9, wherein the method enhances myelination.

11. A modified cell generated by the method of any one of claims 1-10.

12. The cell of claim 11, wherein the cell is selected from a neural stem cell (NSC), oligodendrocyte progenitor cell (OPC), or oligodendrocyte cell.

13. The cell of claim 12, wherein the cell is a NSC or OPC.

14. A cell descended from the modified cell of any one of claims 11-13.

15. A composition comprising the modified cell of any one of claims 11-13, or the cell of claim 14.

16. A method of treating a myelin related disorder in a subject, the method comprising administering to the subject an agent that impairs the activity of Sox6 to arrest developmental transcriptional condensates at an intermediate stage of the oligodendrocyte precursor—oligodendrocyte lineage, thereby accelerating or promoting maturation of said oligodendrocyte precursor to myelin-producing oligodendrocytes.

17. A method of treating a myelin related disorder in a subject, the method comprising administering to the subject a cell generated by the method of any one of claims 1-10, or a cell of any one of claims 11-14, thereby introducing a mature oligodendrocyte cell into the subject.

18. The method of claim 16 or 17, wherein the myelin-related disorder is selected from multiple sclerosis (MS), neuromyelitis optica (NMO), transverse myelitis, chronic inflammatory demyelinating polyneuropathy, Guillain-Barre Syndrome, progressive multifocal leukoencephalopathy (PML), encephalomyelitis (EPL), central pontine myelolysis (CPM), adrenoleukodystrophy, Alexander's disease, Pelizaeus Merzbacher disease (PMD), Wallerian Degeneration, optic neuritis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute dissmeminated encephalitis, Marie-Charcot-Tooth disease and Bell's palsy.

19. The method of any one of claims 16-18, wherein the method alleviates at least one symptom(s) of the subject associated with said myelin related disorder.

20. The method of any one of claims 16-19, wherein the method restores a function of the subject to at least 30%, 40%, 50%, 60%, 70%, 80%, 90%, or about 100% of a control subject without the myelin related disorder, preferably, the function is motor coordination, locomotion, or axon conduction velocity.

21. The method of any one of claims 16-20, wherein the cell is selected from the group consisting of a genetically modified NSC, OPC, and oligodendrocyte.

22. The method of any one of claims 16-21, wherein the subject is a mammal, such as a human.
